SEQUENCE LISTING (46 sequences)

SEQ ID NO: 1 - DNA, 35 bp, Artificial Sequence
  primer VirFLC1F
actgtcgaattcgccagatggagaagagtaatctt

SEQ ID NO: 2 - DNA, 37 bp, Artificial Sequence
  primer VirFLC1R
ctatgcaagcttgagccggagagagagtatagattat

SEQ ID NO: 3 - DNA, 36 bp, Artificial Sequence
  primer VirFLC2F
actgtcctgcagctagccagatggagaagaataatc

SEQ ID NO: 4 - DNA, 37 bp, Artificial Sequence
  primer VirFLC2R
ctatgcaagcttgatatacaacgttcacccttatagg

SEQ ID NO: 5 - DNA, 33 bp, Artificial Sequence
  primer VirFLC3F
actgtcctgcaggctgaaagaagagaatcaggc

SEQ ID NO: 6 - DNA, 33 bp, Artificial Sequence
  primer VirFLC3R
ctatgcatcgatctcagccaagggagtattgag

SEQ ID NO: 7 - DNA, 36 bp, Artificial Sequence; identical to SEQ ID NO: 3
  primer VirFLC4F

SEQ ID NO: 8 - DNA, 36 bp, Artificial Sequence
  primer VirFLC4R
ctatgcaagcttaagagagtgtgaagatatacaacg

SEQ ID NO: 9 - DNA, 37 bp, Artificial Sequence
  primer VirFLC5F
actgtcctgcagccgaagctgataatatagagatgtc

SEQ ID NO: 10 - DNA, 37 bp, Artificial Sequence
  primer VirFLC5R
ctatgcaagcttgggttaaactgacataggttatttg

SEQ ID NO: 11 - DNA, 57 bp, Artificial Sequence
  primer Consensus FLCLong5′
gccctctccgtaactagagctaggaagacagaactaatgttgaagcttgtgatagcc

SEQ ID NO: 12 - DNA, 66 bp, Artificial Sequence
  primer ConsensusFLCLong3′
tggttctcttctttcagcaaattctccttttctttgaggctatcacaagcttcaacattagttctg

SEQ ID NO: 13 - DNA, 32 bp, Artificial Sequence
  primer ConsensusFLCF
actgtcctgcaggccctctccgtaactagagc

SEQ ID NO: 14 - DNA, 32 bp, Artificial Sequence
  primer ConsensusFLCR
ctatgcatcgattggttctcttctttcagcaa

SEQ ID NO: 15 - DNA, 25 bp, Artificial Sequence
  primer CLCV-BGenF
ggatctaccacgatatctaataggc

SEQ ID NO: 16 - DNA, 23 bp, Artificial Sequence
  primer CLCV-BGenR
acagagttagcgacacaaatgtg

SEQ ID NO: 17 - DNA, 24 bp, Artificial Sequence
  primer CLCV-AGenF
aataaagacgacgtctaccacaac

SEQ ID NO: 18 - DNA, 23 bp, Artificial Sequence
  primer CLCV-AGenR
tcttgtgctgtgctttgatagag

SEQ ID NO: 19 - DNA, 23 bp, Artificial Sequence
  primer BnFLC1F
gccagatggagaagagtaatctt

SEQ ID NO: 20 - DNA, 25 bp, Artificial Sequence
  primer BnFLC1R
gagccggagagagagtatagattat

SEQ ID NO: 21 - DNA, 24 bp, Artificial Sequence
  primer BnFLC2F
ctagccagatggagaagaataatc

SEQ ID NO: 22 - DNA, 25 bp, Artificial Sequence
  primer BnFLC2R
gatatacaacgttcacccttatagg

SEQ ID NO: 23 - DNA, 21 bp, Artificial Sequence
  primer BnFLC3F
gctgaaagaagagaatcaggc

SEQ ID NO: 24 - DNA, 21 bp, Artificial Sequence
  primer BnFLC3R
ctcagccaagggagtattgag

SEQ ID NO: 25 - DNA, 24 bp, Artificial Sequence
  primer BnFLC4F
ctagccagatggagaagaataatc

SEQ ID NO: 26 - DNA, 25 bp, Artificial Sequence
  primer BnFLC4R
aagagagtgtgaagatatacaacgc

SEQ ID NO: 27 - DNA, 25 bp, Artificial Sequence
  primer BnFLC5F
ccgaagctgataatatagagatgtc

SEQ ID NO: 28 - DNA, 25 bp, Artificial Sequence
  primer BnFLC5R
gggttaaactgacataggttatttg

SEQ ID NO: 29 - DNA, 23 bp, Artificial Sequence
  primer PDSF
gatataccaaggccagagctaga

SEQ ID NO: 30 - DNA, 226 bp, Brassica napus
  misc_feature (1)...(226): FLC1 fragment
gccagatggagaagagtaatcttgtgcgagccgaagctgataatatggatgtctcaccaggacaaatctccgacatcaatcttccggtaacgctcccactgcttaattagtcacctttaatcggcgaataaataaaatccaaaacatataactaaaacaaacaagatgtgtaattatccccttgtaaagggtgtacgttgtataatctatactctctctccggctc

SEQ ID NO: 31 - DNA, 194 bp, Brassica napus
  misc_feature (1)...(194): BnFLC2 fragment
ctagccagatggagaagaataatcttgcgggagccgaagctgataaaatggagatgtcacctggacaaatctctgacatcaatcgtccggtaactctccgactgctttattagccaccttaagtccaaaacttgtgactaaaaacaaaaataagttatcgaactattcccctataagggtgaacgttgtatatc

SEQ ID NO: 32 - DNA, 260 bp, Brassica napus
  misc_feature (1)...(260): FLC3 fragment
gctgaaagaagagaatcaggctttggctggccagaaggagaagaagaatcttgcgggagccgaagctgataatatggagatgtcacctggacaaatctccgacatcaatcttccggtaactctcccactgcttaattagccaccgttagacggggctgatcaaattaaaaaatccaaaacatacaactaaataaataagctttgttgtttttcacccttgaagggtgcacgttgtatatctcaatactcccttggctgag

SEQ ID NO: 33 - DNA, 206 bp, Brassica napus
  misc_feature (1)...(206): FLC4
ctagccagatggagaagaataatcttgcgggagccgaagctgataaaatggaagtgtcacctggacagatctctgacatcaattgtccggtaactctcccactgctttattagccccttaagtccaaaacttgtgactaaaaacaaaaataagttaccgaactattcccctataagggtgagcgttgtatatcttcacactctctt

SEQ ID NO: 34 - DNA, 236 bp, Brassica napus
  misc_feature (1)...(236): FLC5
ccgaagctgataatatagagatgtcatctggacaaatctccgacatcaatcttcctgtaactctcccgctgcttaattaaccacctttactcggcggttaatcaaaataagaaacatataatctaaagataacctatgtaggttttacttttcgcagcttaattaaccncctttactcggcggttaatcgaaattaaaaacatataattaacaaataacctatgtcagtttaaccc

SEQ ID NO: 35 - DNA, 95 bp, Artificial Sequence
  BnFLC1-5 consensus
gccctctccgtaactagagctaggaagacagaactaatgttgaagcttgttgatagcctcaaagaaaaggagaatttgctgaaagaagagaacca

SEQ ID NO: 36 - DNA, 6224 bp, Artificial Sequence
  Viral vector CaLCuV A Plus FLC1
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggttaacccaagcagcggccttatcaaatttatggatcatacctgcacgaggtgaatagttagcattgcgggacactttagaggtccccgccatagaacgccacggggcatcccgcttaggcatttcgaagtaaagcataaattgaaatcacatgtctttatataggcccacagcgcactcagttagccttaatagctaaccttgtcaggcggcatgtgattggttagatttcatttaatgcgtggtcccacgcaagacaccaaaaaaatcgcggccatccggtaatattattgcggatggccgcttttgcccctggtgttccccagaggcttttagtatatattggacaccaagacaccaaaaacaataggttttagagagagctctcctggcgtcctcccctggagtcctaaatattatttacaaaaatgccacgaaaccctaaatcgtttcgtttagcagcccgaaatatattcttaacatatccccagtgcgacatacccaaagatgaagctcttcagatgcttcaaaccctgtcgtggtcagtcgtcaaacccacatacatcagagtcgcaagagaggaacattcagacgggttcccccatttacactgtctcatccaactatcaggaaagtcgaacatcaaggatgctagatttttcgacatcactcaccccagaaggtctgccaattttcacccaaacattcaggcagccaaagacaccaatgccgtcaagaattacatcaccaaagatggtgattattgtgaatccgggcagtacaaggtgtctgggggtacaaaggcaaataaagacgacgtctaccacaacgccgtcaatgcgggatgtgtggaagaggctctcgcaattataagggctggagatccaaagacgttcattgttagttatcataatgttagagctaacatagagcgactctttactaaggctccggaaccatgggctcctccgtttcaactctcctcctttactaacgtcccggacgagatgagttcatgggcagatgactattttggtcggagtgccgctgcgcgggcggaaagacctattagtatcatagttgaaggtgattcacgaaccggcaagaccatgtgggcgcgtgctttaggaccacataattatttgagtgggcacctcgactttaattcaaaggtcttttcaaataatgcggagtataacgtcattgatgacatagctccgcattatctaaagctaaagcactggaaagagcttattggggcccaaagggactggcaatcaaactgtaagtacggcaagccagttcaaattaaaggtggcataccctcaatcgtgctgtgcaatccaggagaggggagcagttatataagtttcctcaacaaagaggaaaatgcatcactaagagcgtggactaccaaaaatgcaaaattcatcactcttgaagcccccctctatcaaagcacagcacaagattgctaaaaggagagctgtacgccgaagacgcattgatttgaactgcgggtgttctatcttcctccacatcaattgcgcagataatggattcacgcaccggggagagcatcactgtagctcaggcagagaattccgtttttatctgggaggttccaaatcccctatatttcaggatacaacgcgtagaggacccgttgtacaccagaaccaggatctaccacatccaagtccggttcaaccacaacctacggaaagcattggatctccgcaaagcctacttcaacttccaagtctggacgacttcgatgagagcttctgggccgacatatttaagtagattcaaatgccttgtaatgtctcatttagataacctaggtgttattggtattaatcatgtaattagagctgttcgtttcgcaacggacagatcttatgtaactcatgttcacgagaatcatgtaataaatttcaaaatttattaatttgttatcgaatcgtaaaaatagatccgaatttttaatgtcgcatacacaggattagaggcatgagtacatgccatatacaataacagagcgttctccgtatgattctcgtatttccctgcttcttgatggttgtacacaacgtagttattgaccttccaaaaacgcttcaccaacgcctgctcgttactcgcatactgcccaccggttacctttgcgtaaaacttgtgcatgacttgataacgatcacgaagatcgttcttcacagtagcagtgacggtatcgataagcttgagccggagagagagtatagattatacaacgtacaccctttacaaggggataattacacatcttgtttgttttagttatatgttttggattttatttattcgccgattaaaggtgactaattaagcagtgggagcgttaccggaagattgatgtcggagatttgtcctggtgagacatccatattatcagcttcggctcgcacaagattactcttctccatctggcgaattcctgcagcccaacccaagcagcggccttatcaaatttatggatcatacctgcacgaggtgaatagttagcattgcgggacactttagaggtccccgccatagaacgccacggggcatcccgcttaggcatttcgaagtaaagcataaattgaaatcacatgtctttatataggcccacagcgcactcagttagccttaatagctaaccttgtcaggcggcatgtgattggttagatttcatttaatgcgtggtcccacgcaagacaccaaaaaaatcgcggccatccggtaatattattgcggatggccgcttttgcccctggtgttccccagaggcttttagtatatattggacaccaagacaccaaaaacaataggttttagagagagctctcctggcgtcctcccctggagtcctaaatattatttacaaaaatgccacgaaaccctaaatcgtttcgtttagcagcccgaaatatattcttaacatatccccagtgcgacatacccaaagatgaagctcttcagatgcttcaaaccctgtcgtggtcagtcgtcaaacccacatacatcagagtcgcaagagaggaacattcagacgggttcccccatttacactgtctcatccaactatcaggaaagtcgaacatcaaggatgctagatttttcgacatcactcaccccagaaggtctgccaattttcacccaaacattcaggcagccaaagacaccaatgccgtcaagaattacatcaccaaagatggtgattattgtgaatccgggcagtacaaggtgtctgggggtacaaaggcaaataaagacgacgtgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 37 - DNA, 6228 bp, Artificial Sequence
  Viral vector CaLCuV A Plus FLC5
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggttaacccaagcagcggccttatcaaatttatggatcatacctgcacgaggtgaatagttagcattgcgggacactttagaggtccccgccatagaacgccacggggcatcccgcttaggcatttcgaagtaaagcataaattgaaatcacatgtctttatataggcccacagcgcactcagttagccttaatagctaaccttgtcaggcggcatgtgattggttagatttcatttaatgcgtggtcccacgcaagacaccaaaaaaatcgcggccatccggtaatattattgcggatggccgcttttgcccctggtgttccccagaggcttttagtatatattggacaccaagacaccaaaaacaataggttttagagagagctctcctggcgtcctcccctggagtcctaaatattatttacaaaaatgccacgaaaccctaaatcgtttcgtttagcagcccgaaatatattcttaacatatccccagtgcgacatacccaaagatgaagctcttcagatgcttcaaaccctgtcgtggtcagtcgtcaaacccacatacatcagagtcgcaagagaggaacattcagacgggttcccccatttacactgtctcatccaactatcaggaaagtcgaacatcaaggatgctagatttttcgacatcactcaccccagaaggtctgccaattttcacccaaacattcaggcagccaaagacaccaatgccgtcaagaattacatcaccaaagatggtgattattgtgaatccgggcagtacaaggtgtctgggggtacaaaggcaaataaagacgacgtctaccacaacgccgtcaatgcgggatgtgtggaagaggctctcgcaattataagggctggagatccaaagacgttcattgttagttatcataatgttagagctaacatagagcgactctttactaaggctccggaaccatgggctcctccgtttcaactctcctcctttactaacgtcccggacgagatgagttcatgggcagatgactattttggtcggagtgccgctgcgcgggcggaaagacctattagtatcatagttgaaggtgattcacgaaccggcaagaccatgtgggcgcgtgctttaggaccacataattatttgagtgggcacctcgactttaattcaaaggtcttttcaaataatgcggagtataacgtcattgatgacatagctccgcattatctaaagctaaagcactggaaagagcttattggggcccaaagggactggcaatcaaactgtaagtacggcaagccagttcaaattaaaggtggcataccctcaatcgtgctgtgcaatccaggagaggggagcagttatataagtttcctcaacaaagaggaaaatgcatcactaagagcgtggactaccaaaaatgcaaaattcatcactcttgaagcccccctctatcaaagcacagcacaagattgctaaaaggagagctgtacgccgaagacgcattgatttgaactgcgggtgttctatcttcctccacatcaattgcgcagataatggattcacgcaccggggagagcatcactgtagctcaggcagagaattccgtttttatctgggaggttccaaatcccctatatttcaggatacaacgcgtagaggacccgttgtacaccagaaccaggatctaccacatccaagtccggttcaaccacaacctacggaaagcattggatctccgcaaagcctacttcaacttccaagtctggacgacttcgatgagagcttctgggccgacatatttaagtagattcaaatgccttgtaatgtctcatttagataacctaggtgttattggtattaatcatgtaattagagctgttcgtttcgcaacggacagatcttatgtaactcatgttcacgagaatcatgtaataaatttcaaaatttattaatttgttatcgaatcgtaaaaatagatccgaatttttaatgtcgcatacacaggattagaggcatgagtacatgccatatacaataacagagcgttctccgtatgattctcgtatttccctgcttcttgatggttgtacacaacgtagttattgaccttccaaaaacgcttcaccaacgcctgctcgttactcgcatactgcccaccggttacctttgcgtaaaacttgtgcatgacttgataacgatcacgaagatcgttcttcacagtagcagtgacggtatcgataagcttgggttaaactgacataggttatttgttaattatatgtttttaatttcgattaaccgccgagtaaaggnggttaattaagctgcgaaaagtaaaacctacataggttatctttagattatatgtttcttattttgattaaccgccgagtaaaggtggttaattaagcagcgggagagttacaggaagattgatgtcggagatttgtccagatgacatctctatattatcagcttcggctgcagcccaacccaagcagcggccttatcaaatttatggatcatacctgcacgaggtgaatagttagcattgcgggacactttagaggtccccgccatagaacgccacggggcatcccgcttaggcatttcgaagtaaagcataaattgaaatcacatgtctttatataggcccacagcgcactcagttagccttaatagctaaccttgtcaggcggcatgtgattggttagatttcatttaatgcgtggtcccacgcaagacaccaaaaaaatcgcggccatccggtaatattattgcggatggccgcttttgcccctggtgttccccagaggcttttagtatatattggacaccaagacaccaaaaacaataggttttagagagagctctcctggcgtcctcccctggagtcctaaatattatttacaaaaatgccacgaaaccctaaatcgtttcgtttagcagcccgaaatatattcttaacatatccccagtgcgacatacccaaagatgaagctcttcagatgcttcaaaccctgtcgtggtcagtcgtcaaacccacatacatcagagtcgcaagagaggaacattcagacgggttcccccatttacactgtctcatccaactatcaggaaagtcgaacatcaaggatgctagatttttcgacatcactcaccccagaaggtctgccaattttcacccaaacattcaggcagccaaagacaccaatgccgtcaagaattacatcaccaaagatggtgattattgtgaatccgggcagtacaaggtgtctgggggtacaaaggcaaataaagacgacgtgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 38 - DNA, 6080 bp, Artificial Sequence
  Viral vector CaLCuV A Plus FLC Consensus
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggttaacccaagcagcggccttatcaaatttatggatcatacctgcacgaggtgaatagttagcattgcgggacactttagaggtccccgccatagaacgccacggggcatcccgcttaggcatttcgaagtaaagcataaattgaaatcacatgtctttatataggcccacagcgcactcagttagccttaatagctaaccttgtcaggcggcatgtgattggttagatttcatttaatgcgtggtcccacgcaagacaccaaaaaaatcgcggccatccggtaatattattgcggatggccgcttttgcccctggtgttccccagaggcttttagtatatattggacaccaagacaccaaaaacaataggttttagagagagctctcctggcgtcctcccctggagtcctaaatattatttacaaaaatgccacgaaaccctaaatcgtttcgtttagcagcccgaaatatattcttaacatatccccagtgcgacatacccaaagatgaagctcttcagatgcttcaaaccctgtcgtggtcagtcgtcaaacccacatacatcagagtcgcaagagaggaacattcagacgggttcccccatttacactgtctcatccaactatcaggaaagtcgaacatcaaggatgctagatttttcgacatcactcaccccagaaggtctgccaattttcacccaaacattcaggcagccaaagacaccaatgccgtcaagaattacatcaccaaagatggtgattattgtgaatccgggcagtacaaggtgtctgggggtacaaaggcaaataaagacgacgtctaccacaacgccgtcaatgcgggatgtgtggaagaggctctcgcaattataagggctggagatccaaagacgttcattgttagttatcataatgttagagctaacatagagcgactctttactaaggctccggaaccatgggctcctccgtttcaactctcctcctttactaacgtcccggacgagatgagttcatgggcagatgactattttggtcggagtgccgctgcgcgggcggaaagacctattagtatcatagttgaaggtgattcacgaaccggcaagaccatgtgggcgcgtgctttaggaccacataattatttgagtgggcacctcgactttaattcaaaggtcttttcaaataatgcggagtataacgtcattgatgacatagctccgcattatctaaagctaaagcactggaaagagcttattggggcccaaagggactggcaatcaaactgtaagtacggcaagccagttcaaattaaaggtggcataccctcaatcgtgctgtgcaatccaggagaggggagcagttatataagtttcctcaacaaagaggaaaatgcatcactaagagcgtggactaccaaaaatgcaaaattcatcactcttgaagcccccctctatcaaagcacagcacaagattgctaaaaggagagctgtacgccgaagacgcattgatttgaactgcgggtgttctatcttcctccacatcaattgcgcagataatggattcacgcaccggggagagcatcactgtagctcaggcagagaattccgtttttatctgggaggttccaaatcccctatatttcaggatacaacgcgtagaggacccgttgtacaccagaaccaggatctaccacatccaagtccggttcaaccacaacctacggaaagcattggatctccgcaaagcctacttcaacttccaagtctggacgacttcgatgagagcttctgggccgacatatttaagtagattcaaatgccttgtaatgtctcatttagataacctaggtgttattggtattaatcatgtaattagagctgttcgtttcgcaacggacagatcttatgtaactcatgttcacgagaatcatgtaataaatttcaaaatttattaatttgttatcgaatcgtaaaaatagatccgaatttttaatgtcgcatacacaggattagaggcatgagtacatgccatatacaataacagagcgttctccgtatgattctcgtatttccctgcttcttgatggttgtacacaacgtagttattgaccttccaaaaacgcttcaccaacgcctgctcgttactcgcatactgcccaccggttacctttgcgtaaaacttgtgcatgacttgataacgatcacgaagatcgttcttcacagtagcagtgacggtatcgattggttctcttctttcagcaaattctccttttctttgaggctatcacaagcttcaacattagttctgtcttcctagctctagttacggagagggcctgcagcccaacccaagcagcggccttatcaaatttatggatcatacctgcacgaggtgaatagttagcattgcgggacactttagaggtccccgccatagaacgccacggggcatcccgcttaggcatttcgaagtaaagcataaattgaaatcacatgtctttatataggcccacagcgcactcagttagccttaatagctaaccttgtcaggcggcatgtgattggttagatttcatttaatgcgtggtcccacgcaagacaccaaaaaaatcgcggccatccggtaatattattgcggatggccgcttttgcccctggtgttccccagaggcttttagtatatattggacaccaagacaccaaaaacaataggttttagagagagctctcctggcgtcctcccctggagtcctaaatattatttacaaaaatgccacgaaaccctaaatcgtttcgtttagcagcccgaaatatattcttaacatatccccagtgcgacatacccaaagatgaagctcttcagatgcttcaaaccctgtcgtggtcagtcgtcaaacccacatacatcagagtcgcaagagaggaacattcagacgggttcccccatttacactgtctcatccaactatcaggaaagtcgaacatcaaggatgctagatttttcgacatcactcaccccagaaggtctgccaattttcacccaaacattcaggcagccaaagacaccaatgccgtcaagaattacatcaccaaagatggtgattattgtgaatccgggcagtacaaggtgtctgggggtacaaaggcaaataaagacgacgtgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 39 - DNA, 23 bp, Artificial Sequence
  primer PDSR
tcccaagttctccaaataagttc

SEQ ID NO: 40 - DNA, 95 bp, Brassica napus
gccctctccgtaacaagagctaggaagacagaactaatgttgaagcttgtcgagaaccttaaagaaaaggagaagttgctggaagaggagaacca

SEQ ID NO: 41 - DNA, 95 bp, Brassica napus
gccctctccgtaactagagctaggaagacagaactaatgttgaagcttgttgatagcctcaaagaaaaggagaaattgctgaaagaagagaacca

SEQ ID NO: 42 - DNA, 95 bp, Brassica napus
gccctctctctaactagagctaggaagacagaactaatgttgaagcttgttgatagcctcaaagaaaaggagaagctgctgaaagaagagaatca

SEQ ID NO: 43 - DNA, 95 bp, Brassica napus
gccctctccgtaactagagctaggaagacagaactattgttgaagcttgttgatagcctcaaagaaaaggagaaattgctgaaagaagagaacca

SEQ ID NO: 44 - DNA, 95 bp, Brassica napus
gccctctctgtaactagagctcggaaggcagaactaatgttaaagcttgttgaaagtctcaaagaaaaggagaatctgctgaaagaagagaacca

SEQ ID NO: 45 - DNA, 35 bp, Artificial Sequence
  primer PDSF
actgtcctgcaggatataccaaggccagagctaga

SEQ ID NO: 46 - DNA, 35 bp, Artificial Sequence
  primer PDSR
ctatgcatcgattcccaagttctccaaataagttc

(19) United States
(12) Patent Application Publication
(10) Pub. No.: US 2011/0016584 A1
(43) Pub. Date: Jan. 20, 2011

(54) USE OF VIRUS-INDUCED GENE SILENCING (VIGS) TO DOWN-REGULATE GENES IN PLANTS

(75) Inventors: Raul Alvarez-Venegas, Guanajuato (MX); Yongping Zhang, Branpton (CA); Konrad Kraling, Schwedeneck (DE); Lomas Tulsieram, Mississauga (CA)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: PIONEER HI-BRED INTERNATIONAL, INC., Wilmington, DE (US)

(21) Appl. No.: 12/933,270

(22) PCT Filed: Apr. 6, 2009
(86) PCT No.: PCT/US09/39657

§ 371 Date: Sep. 17, 2010

Related U.S. Application Data
(60) Provisional application No. 61/042,815, filed on Apr. 7, 2008.

(51) Int. Cl. A01H 1/00; C12N 15/63

(52) U.S. Cl. 800/278; 435/320.1

(57) ABSTRACT

The present invention provides nucleic acid molecules and methods to down-regulate by virus-induced gene silencing (VIGS) vernalization genes in winter annuals, specifically the Flowering Locus C (FLC) gene in Brassica napus. Down-regulation of FLC allows winter annuals to flower without vernalization or with reduced vernalization. This, in turn, provides a shorter breeding cycling and the opportunity for enhanced genetic gain.

### FIELD OF THE INVENTION

This invention relates to plant molecular biology. In particular the invention relates to virus induced gene silencing (VIGS) in Brassicas and floral induction in winter annuals without the need of vernalization.

### BACKGROUND

Vernalization is the subjection of seeds or seedlings to low temperature in order to hasten plant development and flowering. Vernalization is commonly required for winter annuals such as winter Brassicas and winter wheat. It is believed that seeds and buds of many plants require cold in order to break dormancy and switch from vegetative to reproductive growth (flowering). This mechanism ensures that plants flower during the warmer period of spring or summer. However, from a breeding perspective, the requirement for vernalization is a major impediment in accelerating the rate of genetic gain since the number of breeding cycles per year is restricted. In addition to winter Brassicas and winter wheat, other examples of plants that require vernalization in order to flower include barley, rye, Thlaspi arvense, Daucus carota, some species of Beta vulgaris, and some Arabidopsis ecotypes (Boudry, et al., (2002) Journal of Ecology 90:693-703). Plants that have a vernalization requirement are commonly referred to as ‘winter’ plants, annuals, biennials, lines or varieties.

It has been reported in the scientific literature that in winter annual ecotypes of Arabidopsis thaliana, the level of Flowering Locus C (FLC) activity is proportional to the lateness to flower, that is, loss of function or down-regulation of FLC promotes flowering, while over-expression of FLC delays flowering (Michaels and Amasino, (May 1999) The Plant Cell 11:949-956; Sheldon, et. al., (March 1999) The Plant Cell 11:445-458).

Down-regulation is known to occur in plants using anti-sense technology or co-suppression. More recently, virus-induced gene silencing (VIGS) has been used to down-regulate genes. This technology utilizes plant viruses to express a small fragment of a host target gene in inoculated plants. The replication of the virus vector which includes the small fragment of the host target gene induces a host response that knocks out expression of the endogenous target gene. The fragment of the host target gene on the viral vector must share a certain degree of identity or complementary to the target sequence in order for the silencing to occur. The target sequence may be native or transgenic (Turnage, et al., (2002) Plant J. 30(1):107-114). It has been suggested that the mechanism involved is post-transcriptional and targets RNA molecules in a sequence-specific manner (Smith, et al., (1994) Plant Cell 6:1441-1453). Further, the fact that viruses can both cause and be the targets of gene silencing has suggested that the mechanism is associated with anti-viral plant defense mechanisms (Pruss, et al., (1997) Plant Cell 9:859-868). VIGS can be activated in virally infected plants when a gene, part of a gene, or its RNA is perceived as part of a virus genome or transcript. Further, it is not necessary that all of the viral genome or transcript be present—a portion of the viral genome can be sufficient to induce VIGS.

Geminiviruses are single-stranded DNA viruses that replicate through double-stranded DNA intermediates using the plant DNA replication machinery. Geminiviruses form a large family of plant viruses and are able to infect members of the Brassicaceae. Cabbage Leaf Curl Virus (CaLCuV) is a bipartite geminivirus having single stranded DNA. It is classified in the Begomovirus genus and infects Arabidopsis and Brassica species among others, producing mild symptoms of infection (Turnage, et al., (2002) The Plant Journal 30(1):107-114). Geminiviruses replicate in the nucleus, and foreign DNA can be stably integrated into the viral genome without significantly affecting replication or movement.

The geminiviruses genome is encapsidated in twinned “geminate” icosahedral particles. The encapsidated single stranded DNAs are replicated through circular double stranded DNA intermediates in the nucleus of the host cell. It is believed this is achieved by a rolling circle mechanism. Viral DNA replication involves the expression of only a small number of viral proteins that are necessary either for the replication process itself or facilitates replication or viral transcription. The geminiviruses therefore rely primarily on the machinery of the host to copy their genomes and express their genes.

Geminiviruses are subdivided on the basis of host range in either monocots or dicots and whether the insect vector is a leaf hopper or a white fly species. Monocot-infecting geminiviruses are typically transmitted by arthropods (leaf hoppers) and their genome comprises a single stranded DNA component about 2.7 kb in size (monopartite geminivirus); this type of genome is typified by wheat dwarf virus which is one of a number from the subgroup that has been cloned and sequenced. Most geminiviruses that infect dicot hosts are transmitted by the white fly and possess a bipartite genome comprising similarly sized DNA components (termed A and B).

There appear to be six open reading frames (ORFs) on the two genome components; four are encoded by component A and two by component B. On both components, the ORFs diverge from a conserved 230 nucleotide intergenic region (common region) and are transcribed bidirectionally from double stranded replicative form DNA.

In bipartite genomes, the A component contains viral information necessary for the replication and encapsidation of viral DNA, while the B component encodes functions required for movement of the virus through the infected plant. The A component of these viruses is capable of autonomous replication in plant cells in the absence of component B when inserted as a greater than full length copy into the genome of plant cells (Turnage, et al., (2002) The Plant Journal 30(1):107-114). In monopartite geminivirus genomes, the single genomic component contains all viral information necessary for replication, encapsidation, and movement of the virus.

Brassica is an increasingly important crop. As a source of vegetable oil, Brassica oil presently ranks behind only soybeans and palm in commercial market volume. The oil is used for many purposes such as salad oil and cooking oil. Upon extraction of the oil, the meal is used as a feed source. The most common cultivars of Brassica in the developed world are so-called “double-low” varieties: those varieties low in erucic acid in the oil and low in glucosinolates in the solid meal remaining after oil extraction (i.e., an erucic acid content of less than 2 percent by weight based upon the total fatty acid content, and a glucosinolate content of less than 30 μmol/gram of the oil-free meal). These higher quality forms of Brassica, first developed in Canada, are known as canola. There are primarily three commercial species of Brassica: B. napus, B. rapa and B. juncea. B. napus is the species most widely grown in North America, Europe and Australia. Within B. napus, there are two sub-types: winter and spring varieties. The winter varieties are grown most commonly in Europe, with over 3 million hectares (7.5 million acres) planted in 2004. They are typically planted in the fall and undergo approximately 12 to 14 weeks of vernalization at approximately 4 to 10° C. prior to flowering.

In Arabidopsis accessions the difference between spring and winter growth habit is largely explained by molecular variation at the FRI and FLC loci, while other genes are identified contributing to the annual and biennial behavior in this species (Werner, et al., (2005) PNAS 102(7):2460-2465). In Brassica napus FLC is a main factor controlling the winter growth habit. Tadege, et al., (2001 Plant J. 28(5):545-553) reported that a spring canola variety was delayed in flowering when transformed by conventional stable transformation methods with Arabidopsis AtFLC genes.

### SUMMARY

The Applicants are the first to show the successful use of VIGS technology in Brassicas. Accordingly, an aspect of the invention is to provide a method and use of VIGS in Brassicas.

Another aspect of the invention is to provide the use of the VIGS technology to down-regulate vernalization genes in winter genotypes to induce the transition to flowering without the vernalization requirement normally associated with winter lines, or to reduce the vernalization requirement of winter lines. In particular, the Applicant's teachings include the use of VIGS to down-regulate vernalization genes, for example the Flowering Locus C (FLC), in winter genotypes. VIGS technology is based on an RNA-mediated antiviral defense mechanism which makes use of the silencing machinery that regulates gene expression by the specific degradation of double stranded RNA into short RNA molecules (Ruiz, et al., (June 1998) The Plant Cell 10:937-946; Lacomme, et al., (2003) The Plant Journal 34:543-553). In plants, RNA silencing is known to be involved in different processes, for example development of plant defense against viruses. Thus, when a modified virus, containing fragment(s) of a plant endogenous gene(s) or a sequence shared by a family of genes, is used to inoculate or transform a plant, the silencing mechanism is initiated and the RNA degradation process is turned on to destroy all transcripts from the viral genome and the corresponding host mRNAs. If the modified virus contains a sequence shared by a family of genes, it is possible that the transcripts from the entire family of genes are degraded. Miki, et al., (2005) Plant Physiol. 138:1903-1913 showed that a single inverted repeat (IR) construct could be used to suppress expression of members of a gene family.

An aspect of the invention is to provide a DNA construct comprising (i) a first nucleotide comprising a portion of a viral genome sufficient for viral-induced gene silencing in a winter plant and (ii) a second nucleotide comprising at least a fragment of a vernalization gene or a fragment similar thereto, wherein silencing of an endogenous vernalization gene is induced when the DNA construct is introduced in a winter plant that comprises the endogenous vernalization gene. The vernalization gene can be selected from the group consisting of flowering locus C (FLC), frigida (FRI), vernalization independence 3 (VIP 3), frigida-like 1 (FRL1), FRI-related activators, photoperiod independent early flowing (PIE1), early flowering in short days (EFS), genes related to the PAF1 complex, early flowering 7 (ELF7), early flowering 8 (ELF8), vernalization independence 4 (VIP4), FLC-related repressors, flowering locus M (FLM); MADS affecting flowering 2 (MAF2), MADS affecting flowering 3 (MAF3), MADS affecting flowering 4 (MAF4), ATX1 (Arabidopsis trithorax 1) and wheat vernalization gene 2 (VRN2). For example, the vernalization gene can be BnFLC. Further, the FLC gene can comprise a fragment of a nucleotide selected from the group consisting of GenBank accession numbers: AY036888 (BnFLC1), AY036889 (BnFLC2), AY036890 (BnFLC3), AY36891 (BnFLC4), and AY036892 (BnFLC5). Further still, the second nucleotide can comprise a fragment amplified from primer pairs selected from the group consisting of: SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, and SEQ ID NO: 13 and SEQ ID NO: 14. The second nucleotide can comprise a fragment selected from the group consisting of SEQ ID NO: 30, SEQ ID NO: 31, SEQ ID NO: 32, SEQ ID NO: 33, SEQ ID NO: 34 and SEQ ID NO: 35.

For the DNA construct described above, the viral genome can be a geminivirus genome. For example, the viral genome can be cabbage leaf curl virus (CaLCuV) genome. Further, the geminivirus genome can comprise a nucleotide sequence of GenBank accession number U65529 or U65530, or a portion thereof sufficient to effect VIGS. The DNA construct can be that of FIG. 4 or 5, the sequences of which are provided in SEQ ID NOS: 36, 37 and 38.

For the DNA construct described above, the plant can be selected from the group consisting of winter Brassica, Arabidopsis, wheat, barley, and ryegrass. For example, the plant can be winter Brassica. The winter plant can flower with a reduced requirement for vernalization compared to a corresponding plant which does not contain the vector. For example, a reduced requirement for vernalization could result in a shorter period of subjection to low temperature being required, or a less-extreme low temperature being required. That is, a reduced vernalization requirement could mean a reduction in the time or extent of subjection to low temperature normally required by the plant. In certain cases, the plant flowers without the need for vernalization.

Another aspect of the invention is to provide a method of reducing or eliminating the requirement for vernalization in a winter plant comprising an endogenous vernalization gene, the method comprising the steps: (i) introducing the DNA construct described above into the winter plant; and (ii) growing the winter plant in plant growth conditions, wherein silencing of the endogenous vernalization gene is induced and wherein the silencing of the endogenous vernalization gene reduces or eliminates the requirement for vernalization in the winter plant compared to a corresponding winter plant without the DNA construct. The vernalization gene can be selected from the group consisting of flowering locus C (FLC), frigida (FRI), vernalization independence 3 (VIP 3), frigida-like 1 (FRL1), FRI-related activators, photoperiod independent early flowing (PIE1), early flowering in short days (EFS), genes related to the PAF1 complex, early flowering 7 (ELF7), early flowering 8 (ELF8), vernalization independence 4 (VIP4), FLC-related repressors, flowering locus M (FLM); MADS affecting flowering 2 (MAF2), MADS affecting flowering 3 (MAF3), MADS affecting flowering 4 (MAF4), ATX1 (Arabidopsis trithorax 1), and wheat vernalization gene 2 (VRN2). For example, the vernalization gene can be BnFLC. The DNA construct can be introduced by transient transformation or by stable transformation. The plant can be selected from the group consisting of winter Brassica, Arabidopsis, wheat, barley and ryegrass. For example, the plant can be winter Brassica. The step of introducing the viral silencing vector can be selected from the group consisting of particle bombardment, Agrobacterium-mediated transformation, syringe inoculation, Agrodrench, abrasion of plant surfaces and plasmid inoculation. Using this method, the vernalization requirement can be eliminated or reduced.

Another aspect of the invention is to provide a nucleic acid comprising an FLC gene fragment having a sequence that is at least 90% identical to the full length sequence set forth in any one of SEQ ID NOS: 30 to 35. The gene fragment can have a sequence of any one SEQ ID NOS: 30 to 35.

Another aspect of the invention is to provide a nucleic acid comprising an FLC gene fragment having the sequence set forth in SEQ ID NO: 35.

Another aspect of the invention is to provide a nucleic acid comprising a primer having the sequence set forth in any one of SEQ ID NOS: 1 to 14.

Another aspect of the invention is to provide a nucleic acid comprising a primer having the sequence set forth in any one of SEQ ID NOS: 15 to 18.

Another aspect of the invention is to provide a nucleic acid comprising a primer having the sequence set forth in any one of SEQ ID NOS: 19 to 28.

Another aspect of the invention is to provide use of the nucleic acid of any one of SEQ ID NOS: 30 to 35 to silence an endogenous FLC gene in a plant.

Another aspect of the invention is to provide use of the primer of any one of SEQ ID NOS: 1 to 14 to amplify a fragment of an FLC gene.

Another aspect of the invention is to provide use of the primer of SEQ ID NOS: 15 to 18 to determine viral movement in a plant.

Another aspect of the invention is to provide use of the primer of any one of SEQ ID NOS: 19-28 to assay for down-regulation of an FLC gene in a plant.

Another aspect of the invention is to provide use of the DNA construct described above to down-regulate a vernalization gene in a plant.

Another aspect of the invention is to provide a method of silencing expression of an endogenous plant gene in a Brassica plant cell, comprising introducing a DNA construct into the plant cell, wherein the DNA construct comprises (i) a first nucleotide comprising at least a portion of a CaLCuV genome sufficient to effect VIGS and (ii) a second nucleotide comprising a fragment of the endogenous plant gene, or a fragment similar thereto, wherein introduction of the vector in the plant cell results in silencing of the endogenous gene in the plant cell. The step of introducing the DNA construct can be by transient transformation. The endogenous gene can regulate male fertility. The step of introducing the DNA construct can be by stable transformation.

Another aspect of the invention is to provide a kit for silencing at least one vernalization gene in a plant, comprising: (i) a nucleic acid described above and (ii) instructions for silencing the vernalization gene in the plant.

Another aspect of the invention is to provide a kit for amplifying an FLC gene in a plant, comprising: (i) the nucleic acid described above and (ii) instructions for amplifying the FLC gene.

Another aspect of the invention is to provide a kit for assaying for viral movement in a plant, comprising: (i) the nucleic acid described above and (ii) instructions for assaying for viral movement in the plant.

Another aspect of the invention is to provide a kit for assaying for down-regulation of an FLC gene in a plant cell, comprising: (i) the nucleic acid described above and (ii) instructions for assaying for down-regulation of the FLC gene in the plant.

Any of the kits described above can further comprise buffers and reagents. Further, the invention also provides a combination kit comprising at least two of the kits described above.

Another aspect of the invention is to provide a method for the commercial reduction of the requirement for vernalization in a population of winter plants comprising an endogenous vernalization gene, the method comprising the steps: (i) introducing the DNA construct described above into the population of winter plants; and (ii) growing the population of winter plants in plant growth conditions, wherein silencing of the endogenous vernalization gene is induced and wherein the silencing of the endogenous vernalization gene reduces or eliminates the requirement for vernalization in the population of winter plants compared to a corresponding population of winter plants without the DNA construct. The vernalization gene can be selected from the group consisting of flowering locus C (FLC), frigida (FRI), vernalization independence 3 (VIP 3), frigida-like 1 (FRL1), FRI-related activators, photoperiod independent early flowing (PIE1), early flowering in short days (EFS), genes related to the PAF1 complex, early flowering 7 (ELF7), early flowering 8 (ELF8), vernalization independence 4 (VIP4), FLC-related repressors, flowering locus M (FLM); MADS affecting flowering 2 (MAF2), MADS affecting flowering 3 (MAF3), MADS affecting flowering 4 (MAF4), ATX1 (Arabidopsis trithorax 1) and wheat vernalization gene 2 (VRN2). The vernalization gene can be BnFLC. The population of winter plants can be selected from the group consisting of winter Brassica, Arabidopsis, wheat, barley and ryegrass. For example, the population of winter plants can be winter Brassica. The step of introducing the DNA construct can be selected from the group consisting of Agrodrench and abrasion of plant surfaces.

Another aspect of the invention is to provide a host cell comprising the DNA construct described above. The host cell can be a plant cell.

Another aspect of the invention is to provide a plant comprising the DNA construct described above. The plant can be selected from the group consisting of winter Brassica, Arabidopsis, wheat, barley and ryegrass. For example, the plant can be winter Brassica.

Another aspect of the invention is to provide a population of winter Brassica plants comprising the DNA construct described above.

These and other features of the applicant's teachings are set forth herein.

### BRIEF DESCRIPTION OF THE DRAWINGS

The skilled person in the art will understand that the figures described below are for illustration purposes only. The figures are not intended to limit the scope of the applicant's teachings in any way.

FIG. 1 shows the vector maps of plasmids containing the CaLCuV A (A) and CaLCuV B (B) viral DNA components.

FIG. 2 shows the cDNA sequences of a fragment of BnFLC1 (SEQ ID NO: 40), BnFLC2 (SEQ ID NO: 41), BnFLC3 (SEQ ID NO: 42), BnFLC4 (SEQ ID NO: 43), and BnFLC5 (SEQ ID NO: 44) which spans the interval indicated, and compares these sequences with the consensus sequence of this fragment (SEQ ID NO: 35).

FIG. 3 shows the vector maps of pBSIIKS (A) and pBSIIKS+plus BnFLC1 (B).

FIG. 4 shows the vector maps of the plasmids containing CaLCuV A plus FLC1 (A) and FLC5 (B) respectively.

FIG. 5 shows the vector map of the plasmid containing the CaLCuV A plus the FLC consensus sequence.

FIG. 6 shows the vector map of the PHP 13184.

FIG. 7 shows the vector map of the plasmid containing the CaLCuV A and the phytoene desaturase gene (PDS) from Brassica napus (Genbank Accession #CD827969 submitted by Genoplante 2003)

FIG. 8 shows the steps in the method for biolistic transformation and the flowering without vernalization of an FLC-Consensus transformed winter canola plant of the first biolistic transformation experiment 9 weeks post-transformation. The steps shown clockwise include (i) growing seedlings in vitro, (ii) bombarding seedlings, (iii) growing the transformed seedlings and observing changes in phenotype, (iv) observing bolting without vernalization and (v) allowing the transformed plant to flower.

FIG. 9 is a photograph 14-weeks post transformation in the second biolistic transformation of FLC-Consensus transformed winter canola plants (#5, #20 and #1) showing flowering without vernalization.

FIG. 10 is a photograph of two gels A and B, showing the absence of the A and B components in T1 progeny of FLC-consensus transformed plants. (A) PCR reactions in T1 progeny to identify viral A component. Primers used: CLCV-A, SEQ ID NOS: 17 and 18 (PCR size 665 bp). (B) PCR reactions in T1 progeny to identify viral B component. Primers used: CLCV-B, SEQ ID NOS: 15 and 16 (PCR size 773 bp).

### DEFINITIONS

As used herein, “endogenous” plant gene refers to a gene integrated into the chromosomal DNA of the plant genome. Endogenous genes include those that occur naturally in the plant genome, as well as those stable exogenous genes artificially introduced by genetic transformation.

As used herein, “FLC” means flowering locus C.

The term “introduced” when referring to a heterologous or isolated nucleic acid refers to the incorporation of a nucleic acid into a eukaryotic or prokaryotic cell. The nucleic acid may be incorporated into the genome of the cell (e.g., chromosome, plasmid, plastid or mitochondrial DNA), converted into an autonomous replicon, or transiently expressed (e.g., transfected mRNA). The term includes such nucleic acid introduction means as “transfection,” “transformation” and “transduction.”

As used herein “silenced” or “gene silencing” refers to a reduction in the expression product of a target gene. Silencing may occur at the transcriptional or post-transcriptional level. Silencing may occur anywhere throughout the plant. Silencing may be complete, in that no final gene product is produced, or partial, in that a reduction in gene product occurs. For example, the gene product can be reduced by 10 to 100%.

As used herein, “vernalization” means the subjection of seeds, seedlings, or plants to low temperature in order to break dormancy and switch from vegetative to reproductive growth (flowering).

As used herein, “vernalization genes” or “genes involved in vernalization” are those genes that are expressed in winter plants that delay flowering until the winter plants are subject to low temperature in order to break dormancy and switch from vegetative to reproductive growth (flowering). Of particular interest in the present invention are those genes whose expression activates or causes the vernalization requirement. The present invention includes methods to down-regulate the genes that cause or activate the vernalization requirement, and therefore allow the plant to flower without the need for vernalization. Examples of these genes include, but are not limited to, flowering locus C (FLC), frigida (FRI), vernalization independence 3 (VIP 3), frigida-like 1 (FRL1), photoperiod independent early flowing (PIE1), early flowering in short days (EFS), and any other genes related to the PAF1 complex, including early flowering 7 (ELF7), early flowering 8 (ELF8), and vernalization independence 4 (VIP4). Other genes include FLC-related repressors, for example flowering locus M (FLM) and FRI-related activators. In addition, FLC relatives include for example, MADS affecting flowering 2 (MAF2), MAF3, and MAF4. Another example is ATX1 (Arabidopsis trithorax 1). Another example includes the wheat vernalization gene, VRN2, a dominant repressor of flowering that is down-regulated by vernalization (Yan, et al., (2004) Science 303(5664):1640-1644). The wheat vernalization gene, VRN2, can be considered a functional homolog of Brassica FLC genes because it is also down-regulated by vernalization. Other functional homologs are also included in the scope of the invention. Structural homologues of the vernalization genes are also included in the scope of the invention. “Similar” sequences or fragments that show a sufficient percent identity or a sufficient percent complementarity to the sequences described above and are capable of affecting vernalization are also included in the scope of the invention. For example, the “similar” sequences can be between about 50 and 100% identical to the sequences described above. In addition, the similar sequences can be between about 50% to 100% complementary to the sequences described above. The term “between about 50 to 100%” includes all possible integers, for example 51%, 52%, 99%, etc.

As used herein, “VIGS” means virus-induced gene silencing.

As used herein, “viral silencing vector” means a DNA construct comprising (i) a sufficient portion of a viral genome to induce VIGS and (ii) a nucleotide sequence that is similar (i.e., a sequence that has a sufficient percent identity or a sufficient percent complementarity to effect down regulation) to at least a fragment of a target gene, wherein the target gene is down-regulated when the viral silencing vector is introduced into a cell. For example, in order to effect VIGS in a plant, the portion of the viral genome required to effect VIGS may include that portion responsible for viral movement and viral replication in the plant. As is known to those skilled in the art, each virus/host combination should be optimized for producing effective silencing vectors. In the present invention, the viral genome includes all genes except those encoding the coat protein. However, it is to be understood that other optimized vectors can be used and are included within the scope of the applicant's teachings. For example, the silencing vector may include the origin of replication, the genes necessary for replication in a plant cell, and one or more nucleotide sequences with similarity to one or more target genes. The vector may also include those genes necessary for viral movement. In the case of bipartite viruses, for example geminiviruses, the A and B components may be carried in the same silencing vector. Alternatively, the plant may be transformed with both components on separate vectors. Further, in one example, the A genome component of a geminivirus (which replicates autonomously) was shown to be sufficient for VIGS, as was the B component (WO 01/94694 and US Patent Application Publication Number 2002/0148005, both of which are incorporated herein by reference). Other silencing vectors are disclosed in U.S. Pat. No. 6,759,571 and US Patent Application Publication Number 2004/0019930, both of which are herein incorporated by reference. The nucleotide sequence that is similar to at least a fragment of a target gene may replace any coding or non-coding region that is nonessential for the present purposes of gene silencing, may be inserted into the vector outside the viral sequences, or may be inserted just downstream of an endogenous viral gene, such that the viral gene and the nucleotide sequence are cotranscribed. The size of the nucleotide sequence that is similar to the target gene may depend on the site of insertion or replacement within the viral genome. Accordingly, there are many ways of producing silencing vectors, as known to those skilled in the art.

### DESCRIPTION OF THE VARIOUS EMBODIMENTS

Vernalization is the subjection of seeds, seedlings or plants to low temperature in order to break dormancy and switch from vegetative to reproductive growth (flowering). This mechanism ensures that plants flower during the warmer period of spring or summer. From a breeding perspective, the requirement for vernalization is a major impediment in accelerating the rate of genetic gain since the number of breeding cycles per year is restricted. Vernalization is a mitotically stable process. Explants regenerated from a vernalized plant can flower without further vernalization. However, the vernalization process is reset after meiosis and the new generation requires normal vernalization in order to flower (Henderson, et al., (2003) Annu. Rev. Genet. 37:371-392).

An aspect of the applicant's teaching is to transiently down-regulate vernalization genes in winter annuals using VIGS to reduce or eliminate the requirement for vernalization.

It has been reported in the literature that in winter annual ecotypes of Arabidopsis thaliana, the level of Flowering Locus C (FLC) activity is proportional to the lateness to flower, that is, loss of function or down-regulation of FLC promotes flowering, while over-expression of FLC delays flowering (Michaels and Amasino, (May 1999) The Plant Cell 11:949-956). Originally two genes, FRI (FRIGIDA) and FLC were thought to be involved in vernalization-related flowering initiation. Later it was found that FLC can function independently without the presence of FRI though FRI strongly enhances the expression of FLC (Michaels and Amasino, (2001) The Plant Cell 13:935-941). It has been shown that FLC, a MADS-box transcription factor, plays a central role in flowering initiation through repressing a set of genes termed floral pathway integrators: FT, SOC1 and LFY. Suppression of FLC expression consistently promotes flowering in many different plants and there is a quantitative relation between the FLC mRNA levels and the timing of flowering (Simpson, et al., Science 296:285-89). Therefore the effect of FLC is dosage-dependent.

Three additional genes of the vernalization pathway have been identified and cloned recently: VIN3 (vern-insensitive 3), and VRN1 and VRN2 (vernalization) (Wood, et al., (2006) PNAS 103:39; Bastow, et al., Nature 427:164-167; Sung, et al., (2004) Nature 427:159-164). Their functions give a better molecular understanding of the vernalization pathway. VIN3 is the first gene that is activated by vernalization. VIN3 mRNA accumulates during vernalization, but becomes undetectable within 3 days after the return to warm temperature. VIN3 expression is necessary for deacetylation of FLC, which in turn leads to histone methylation and the formation of mitotically stable heterochromatin at the FLC chromatin site by a process involving VRN1 and VRN2. VRN1 and VRN2 are expressed constitutively before, during and after vernalization. It is believed that they function downstream of VIN3 and maintain stability of the transiently acting complex that leads to FLC chromatin silencing and hence FLC repression. This is thought to be the molecular basis of the “winter” memory in the vernalized plants where the FLC expression is kept low even after vernalization and return to warm temperature. After meiosis, the prevernalization state of the FLC chromatin is reset. This would explain why in the next generation the normal vernalization requirement is restored. An additional gene, Arabidopsis trithorax 1 (ATX1) was shown to regulate FLC (Pien, et al., (2008) The Plant Cell Preview Online Publication).

In winter Brassica, five FLC genes have been isolated, named BnFLC1 to 5 (Tadege, et al., (2001) The Plant Journal 28(5):545-553). The 5 FLC genes from B. napus and the Arabidopsis FLC gene have similar functions. Each of five BnFLC genes can function to repress flowering in transgenic Arabidopsis. In addition, spring canola was delayed in flowering when transformed with the Arabidopsis AtFLC gene (Tadege, et al., (2001) Plant J. 28:545-553). Recently four FLC homologues have been isolated in B. rapa and three in B. oleracea (Schramz, et al., (2002) Genetics 162:1457-68). All these FLC genes from Arabidopsis or Brassica species belong to a large multigene family of MADS-box transcription factors. The MADS box is a highly conserved sequence motif found in a family of transcription factors. The conserved domain was recognized after the first four members of the family, which were MCM1, AGAMOUS, DEFICIENS and SRF (serum response factor). The name MADS was constructed from the “initials” of these four “founders”. The FLC genes from Arabidopsis or Brassica all have a MADS-box domain at the 5′ terminus and are conserved at the coding region.

Taken together, all scientific evidence has shown that the FLC gene is a key player for delayed flowering and vernalization requirement in winter annuals. It is a good first target for gene silencing to reduce or eliminate the vernalization requirement in winter types.

One of the main objectives of the VIGS technology developed and used in this invention is to transiently eliminate or reduce the need for vernalization and promote flowering when needed in a winter plant. This has not been done before. When there is no longer a need to eliminate or reduce vernalization in the winter plant, the plant reverts to its normal state in the next generation (i.e., the plant will have a requirement for vernalization to promote flowering). For example, one could eliminate or reduce the requirement for vernalization when breeding new winter varieties by decreasing the length of each generation and therefore increasing the rate of genetic gain. As explained earlier, the new varieties developed revert to their normal requirement for vernalization in the next generation. In this way, the new variety maintains its “winter” phenotype and is grown in its traditional geographical regions, for example, in Europe. Europe is a major winter canola market and non-genetically modified market. Further, the new variety is not considered transgenic because there is no integration of new DNA in the plant genome and therefore the genetic code of the plant has not been altered. Accordingly, one aspect of the invention is to transiently silence the FLC genes in a reliable and efficient manner to the extent that the silencing can be applied on a routine basis in a winter breeding program during germplasm and product development phases.

Another aspect of the invention is to apply this technology on a larger scale, for example in seed production. This allows for production of seeds in non-winter environments. For example, the plants can be grown in off season locations, or the seeds can be planted in spring in winter locations. The plants can be grown outdoors or indoors year round without requiring vernalization.

Using viral vectors to silence an endogenous plant gene may involve cloning into the viral genome, without significantly compromising viral replication and movement, a nucleotide fragment sharing a certain percentage identity or complementarity to the endogenous plant gene. The principle and detailed protocol regarding the VIGS system have been described (Dinesh-Kumar, et al., (2003) Methods in Mol. Biol. 236:287-94; Lu, et al., (2003) Methods 30:296-303). Several different RNA and DNA plant viruses have been modified to serve as vectors for gene expression. These RNA viruses, such as TMV (tobacco mosaic virus), PVX (potato virus X), and TRV (tobacco rattle virus), have been used to silence many different target genes (Angell, et al., (1999) Plant J. 20:357-62; Kumagai, et al., (1995) PNAS 92:1679-83; MacFarlane, et al., (2000) Virology 267:29-35) and could be used for protein expression. Though DNA viruses, limited to Geminiviridae, have not been extensively used as expression vector, tomato golden mosaic virus (TGMV) and cabbage leaf curl virus (CaLCuV) have been used to generate silencing vectors and silenced both transgenes and native genes in tomato and Arabidopsis (Peele, et al., (2001) Plant J. 27:357-66; Turnage, et al., (2001) Plant J. 107:14). As is known to those skilled in the art, each virus/host combination should be optimized for producing effective silencing vectors. In the present invention, the viral genome includes all genes except those encoding the coat protein. However, it is to be understood that other optimized vectors can be used and are included within the scope of the applicant's teachings. For example, the silencing vector may include the origin of to replication, the genes necessary for replication in a plant cell, and one or more nucleotide sequences with similarity to one or more target genes. The vector may also include those genes necessary for viral movement. In the case of bipartite viruses, for example geminiviruses, the A and B components may be carried in the same silencing vector. Alternatively, the plant may be transformed with both components on separate vectors. In one example, the A genome component of a geminivirus (which replicates autonomously) was shown to be sufficient for VIGS, as was the B component (WO 01/94694 and US Patent Application Publication Number 2002/0148005, both of which are incorporated herein by reference). These references indicate that the A genome (AL1, AL2 and/or AL3) or the B genome (BR1 and/or BL1) may be used as a silencing vector. Other silencing vectors are disclosed in U.S. Pat. No. 6,759,571 and US Patent Application Publication Number 2004/0019930, both of which are herein incorporated by reference. WO 01/94694 (incorporated herein by reference) discloses the locations of the geminivirus genome where the nucleotide sequences may be inserted. For example, the nucleotide sequence that is similar to at least a fragment of a target gene may replace any coding or non-coding region that is nonessential for the present purposes of gene silencing, may be inserted into the vector outside the viral sequences, or may be inserted just downstream of an endogenous viral gene, such that the viral gene and the nucleotide sequence are cotranscribed. For example, the nucleotide sequence may be inserted in the common region of the viral genome, however it is preferred that the nucleotide sequences not be inserted into or replace the Ori sequences or the flanking sequences that are required for viral DNA replication. The size of the nucleotide sequence that is similar to the target gene may depend on the site of insertion or replacement within the viral genome.

VIGS technology, through the down-regulation of vernalization genes, is used herein to (i) eliminate or reduce the long periods of vernalization, (ii) increase the number of breeding cycles per year, to accelerate genetic recombination and (iii) increase rate of genetic gain. This is important because it will reduce the length of time necessary to breed winter plants, which currently is a very lengthy and costly process. For example, in order to breed one cycle of a Brassica napus winter canola line, a full 9-10 months are required. The seed is planted and the seedling grows to approximately the 4-5 true leaf stage (6 weeks), then the plants are vernalized at approximately 4° C. to 10° C. for approximately 12 to 14 weeks. After vernalization, the plants bolt, flower, set seed and are harvested (20 weeks). Accordingly, approximately 38 weeks are required for one cycle of winter canola breeding. In comparison, a Brassica napus spring canola plant can complete a life cycle in 13-20 weeks, and it is not uncommon to grow three spring generations in one year, with the use of winter nurseries and greenhouses during the cold season. The ability to transiently (generation specific) reduce or eliminate the vernalization requirement in winter plants when breeding new varieties would allow similar advantages in winter breeding. This would result in a time savings and therefore a cost savings to breed winter plants. In addition, genetic gains in winter lines would occur more readily.

VIGS technology can be used and exploited to silence almost any gene in any developmental or metabolic pathway, and not only those genes involved in the vernalization pathway.

VIGS has not been shown in Brassica prior to this invention. VIGS in Brassica can be used to down-regulate, in a transient manner, many genes other than FLC. This strategy can be applied to Brassica to facilitate breeding or production efficiency. For example, one could down-regulate the genes involved in male reproduction in female lines so that they are male sterile during seed production. In this way, the traditional pollination control systems, like cytoplasmic male sterility (CMS), genic male sterility (GMS), self-incompatibility (SI), etc. which complicate breeding and increase the time and effort to breed new varieties, are no longer needed. Further, there would be no need to breed male and female lines separately. Any line could potentially be used as a female. Although two examples are provided (e.g., modulation of vernalization and male reproduction), it is to be understood that VIGS can be used to down-regulate any gene and to control any metabolic pathway. It can also be used as a tool in functional genomics, especially for identification of genes critical to cell or plant survival, for which stable loss-of-function mutations are lethal.

Cabbage Leaf Curl Virus (CaLCuV) is a member of the geminiviruses and infects Arabidopsis and Brassica, among other species. It has been shown that the viral coat protein of CaLCuV, encoded by AR1, is dispensable when mechanical or agroinoculation methods were used to infect host plants, and that these AR1-deleted vectors are able to propagate (Turnage, et al., (2002) The Plant Journal 30(1):107-114). Plasmids containing the cloned Cabbage Leaf Curl Virus (CaLCuV) viral DNA genome (A and B components, GenBank accession number U65529 and U65530, respectively) were obtained from Ernest Hiebert, University of Florida. CaLCuV A component consists of the cabbage leaf curl virus coat protein (AV1), replicase associated protein (AC3), transactivator protein (AC2), replicase associated protein (AC1) and AC4 genes. CaLCuV B component has the cabbage leaf curl virus nuclear shuttle movement protein (BV1) and movement/pathogenicity protein (BC1) genes. VIGS based vectors do not require promoters and other regulatory elements because the viral genome provides all the elements necessary for expression in a plant cell. The experiments were done using sense sequences cloned into the viral genome to create the VIGS vectors. It is to be understood that antisense sequences can also be used in VIGS vectors and are included in the applicant's teachings.

The transcribed RNA generally includes a sequence (a target sequence) which is complementary to a sequence in a target gene, either in the sense or antisense orientation, or a sequence which has sufficient complementarity to a target sequence for down-regulation to occur. Although Applicants are not bound by any theory, it is currently believed that sense and antisense regulation involve hybridization between molecules which are sufficiently complementary to hybridize under conditions within a cell. Plant virus-based vectors carrying plant sequences with sufficient complementarity to the endogenous plant genes trigger gene silencing through a homology-dependent RNA degradation mechanism commonly referred to as RNA silencing. The dsRNA replication intermediate derived from the virus would be processed so that the small interference RNA (siRNA) in the infected cell would correspond to parts of the viral vector genome, including any nonviral insert (Baulcombe, (2002) Current Biol. 12(3):R84). If the insert is from a host gene, the siRNAs would target the RNase complex to the corresponding host mRNA and the symptoms in the infected plants would reflect the loss of function of the host gene. A targeting sequence in the DNA construct may be a wild-type sequence, a mutant, derivative, variant or allele. The sequence need not include an open reading frame or specify any RNA that would be translatable. The sequence may be inserted in either orientation for sense or anti-sense regulation. As stated above, there should be sufficient complementarity for the sequences to hybridize. There is good silencing even if there is about 5% or 10% mismatch in the initiator of the silencing and the target RNA (Baulcombe (2002) Current Biology Vol 12 No 3).

Further, the DNA construct may comprise more than one targeting sequence for inactivation of more than one target gene.

A vector is provided comprising the construct to be used in the transformation of one or more plant cells. The vector can be used for transient transformation, or a related vector (for example, one carrying the nucleotide sequences of the present invention) can also be used for stable transformation. Accordingly, another aspect of the invention is the stable transformation of winter annuals to reduce or eliminate the vernalization requirement. Methods and constructs for stable transformation are known to those skilled in the art. For stable transformation, the construct is inherited from one generation of the transformed cell to the next. For stable transformation, the genetically transformed plant cell can be regenerated by methods known to those skilled in the art to produce a transgenic plant, which can then produce subsequent generations of plants containing the construct. Accordingly, a construct or vector can be used in the production of stably transformed transgenic plants. As described in WO 01/94604 (herein incorporated by reference) and known to those skilled in the art, a plant cell may be stably transformed with a geminivirus A component (or geminivirus AL1, AL2 and/or AL3 genes), and then inoculated with a silencing vector comprising a geminivirus B genomic component (or geminivirus BR1 and/or BL1 genes). The stably incorporated replication genes from the A component (or A genes) will support the replication of the silencing vector comprising the B component (or B genes). The converse is also possible (stably transforming with the B component or B genes and introducing a silencing vector with the A component or A genes).

As stated above, VIGS based vectors do not require promoters and other regulatory elements because the viral genome provides all the elements necessary for expression in a plant cell. However, the DNA construct may comprise a heterologous or non-viral promoter or other regulatory sequence operably linked to the DNA sequence. This is also considered within the scope of the applicants' teachings. The function of the promoter is to ensure that the DNA is transcribed into RNA containing the viral sequences and the sequence that is similar to the target sequence. By “promoter” is meant a sequence of nucleotides from which transcription may be initiated of DNA operably linked downstream (i.e., in the 3′ direction on sense strand of double stranded DNA). “Operably linked” means joined as part of the same nucleic acid molecule, suitably positioned and orientated for transcription to be initiated from the promoter.

Any promoter can be used as is known to those skilled in the art. A promoter includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses, and bacteria which comprise genes expressed in plant cells such Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions. For example, constitutive promoters include the 35S from Cauliflower Mosaic Virus (CaMV) and the nopaline synthase (nos) promoter from Agrobacterium.

Expression comprises transcription of the heterologous DNA sequence into mRNA. Regulatory elements ensuring expression in eukaryotes are well known to those skilled in the art. In the case of eukaryotic cells, they comprise polyA signals ensuring termination of transcription and stabilization of the transcript. The polyA signals commonly used include that of the 35S RNA from CaMV and that of the nos gene from Agrobacterium. Other regulatory elements can include transcriptional and/or translational; enhancers, introns, and others as is known to those skilled in the art.

Any methods of inoculation or transformation may be used as is known to those skilled in the art. The delivery methods for VIGS constructs include but are not limited to, mechanical injection of in vitro transcribed RNA or extracts from infected plants, Agrobacterium (Agro)-inoculation, inoculation by gentle abrasion of the surfaces of the leaves with carborundum and plasmid DNA (“plasmid inoculation”), and microprojectile bombardment. Mechanical injection is time consuming but can increase the efficiency of silencing in certain hosts such as Arabidopsis (Ratcliff, et al., (2001) Plant J. 25:237-45). Agro-inoculation is the most popular and has been developed for both DNA and RNA viruses (Schob, et al., (1997) Mol. Gen. Genet. 256:581-85). Agro-inoculation is more feasible for large-scale production application and less time consuming. Tobacco, tomato, and barley VIGS vectors have been developed and shown extensive silencing using Agro-inoculation. Specifically, TRV-derived VIGS vector/Agro-inoculation is becoming the dominant combination for many investigators. Inoculation by gentle abrasion of the surfaces of the leaves with carborundum and plasmid DNA is described in Uhde, et al., (2005) Arch. Virol. 150:327-340. Microprojectile bombardment of plasmid DNA-coated tungsten or gold micron-sized particles has been extremely useful for DNA virus-based VIGS vector (Muangsan, et al., (2004) Plant J. 38:1004-14).

Ryu, et al., (WO 2005/103267) describes a method of VIGS via agroinoculation by drenching roots of the plants in a suspension of Agrobacterium (Agrodrench).

Suitable plants for use in the present methods include any plant with a vernalization requirement, including, but not limited to, Graminaceae and Brassicaceae species. Other examples include ryegrass (Lolium perenne L.), diploid wheat (Triticum monococcum), barley (Hordeum sp.), alfalfa, clover, etc. The FLC gene or gene family is not present in all winter varieties, and the applicant's teaching is not limited to the down-regulation of the FLC gene. For example, in winter wheat and barley FLC is not present. Vernalization in wheat and barley is achieved by inducing the expression of a gene which is repressing a flowering repressor, VRN2. The VRN2 gene in wheat is not the same as the VRN2 gene in Arabidopsis. The wheat VRN2 (AY485968) has the CCT motif, while the Arabidopsis (NP_{—}001078563) and the canola (e.g., AAK70219) FLC proteins have the MADS domain. These proteins are down-regulated by vernalization to promote flowering. Accordingly, they are functional homologs. Down-regulation of VRN2 by stable transformation of winter wheat resulted in spring lines which did not require vernalization to flower (Yan, et al., (2004) Science 303:1640). VIGS technology can be used in wheat and barley to down-regulate the flowering repressor gene (wheat VRN2) to induce flowering in a transient manner. The down-regulation of wheat VRN2 and similar genes involved in vernalization are also included within the scope of the applicant's teaching.

### EXAMPLES

Aspects of the applicant's teachings may be further understood in light of the following examples, which should not be construed as limiting the scope of the present teachings in any way.

### Example 1

### Cloning

The five Brassica napus FLC cDNA's (BnFLC1-5) were disclosed by Tadege, et al., (The Plant Journal (2001) 28(5):545-553) and are publicly available through GenBank accession numbers: BnFLC1 AY036888, BnFLC2 AY036889, BnFLC3 AY036890, BnFLC4 AY36891, BnFLC5 AY036892.

In addition, partial genomic sequences found in Brassica oleracea and Brassica rapa are publicly available through the following GenBank accession numbers:

1) Brassica oleracea truncated FLC2 gene GenBank accession number DQ222850

2) Brassica oleracea truncated FLC3 gene GenBank accession number AY115673

3) Brassica oleracea truncated FLC5 gene GenBank accession number AM231526

4) Brassica rapa truncated FLC1 gene Gen Bank accession number AY115678

5) Brassica rapa truncated FLC3 gene Gen Bank accession number AY115677

6) Brassica oleracea truncated FLC5 gene GenBank accession number AY115675

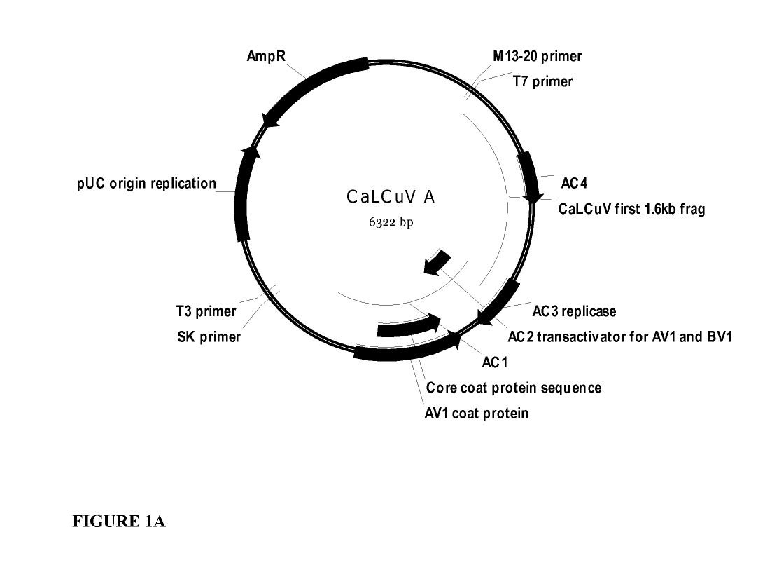

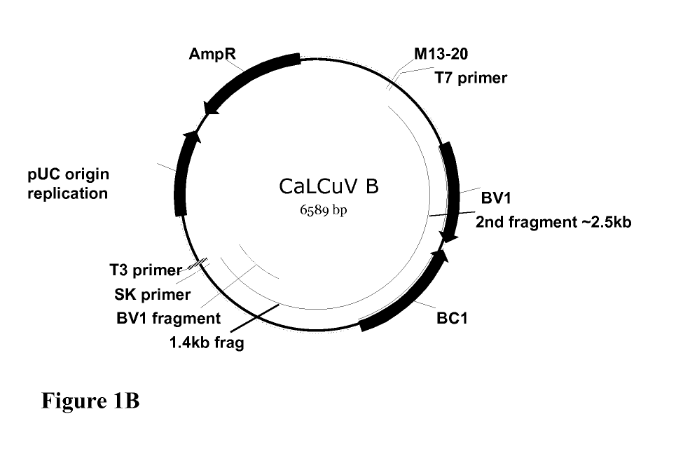

Plasmids containing the cloned Cabbage Leaf Curl Virus (CaLCuV) viral DNA genome (A and B components) were provided by the University of Florida as pUC19 derivative vectors (FIG. 1) (Abouzid, et al., (1992) Phytopathology 82:1070-1070).

With the sequence information from GenBank, specific primers for RT-PCR reactions (following Invitrogen™ instructions, catalog #28025-013) were designed to amplify by RT-PCR, and to clone in a sense orientation only, the different FLC gene fragments from Brassica napus winter line “Express”. The primers cover the 3′-end and the 3′-UTR region of each gene, which is the reason that RT-PCR reactions were chosen. In this way, the primers were specific to each sequence. The primers are shown in Table 1. The PCR conditions were 25 cycles for 30 seconds at 95° C., 30 seconds at 58° C., 30 seconds at 72° C. The nucleotide sequences of the fragments obtained by PCR are shown in SEQ ID NOs: 30-34.

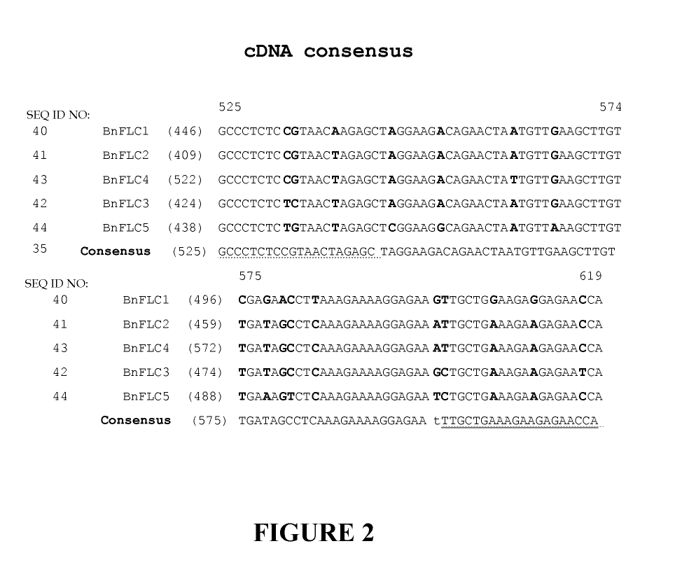

These fragments, named BnFLC1 to BnFLC5 (SEQ ID NO: 30 to 34), were cloned into vectors as described below. A consensus sequence (SEQ ID NO: 35) from another region of the BnFLC1 to 5 genes was identified as shown in FIG. 2. It is to be understood that other fragments and other sized fragments are within the scope of the application. The fragments can be approximately between 50 and 800 nucleotides in length.

TABLE 1
Cloning primers to amplify BnFLC1-5 fragments used
in the viral vectors:

| | | |
|---|---|---|
| VirFLC1F: | ACTGTCgaattcGCCAGATGGAGAAGAGTAATCTT; | SEQ ID NO: 1 |
| VirFLC1R: | CTATGCaagcttGAGCCGGAGAGAGAGTATAGATTAT; | SEQ ID NO: 2 |
| VirFLC2F: | ACTGTCctgcagCTAGCCAGATGGAGAAGAATAATC; | SEQ ID NO: 3 |
| VirFLC2R: | CTATGCaagcttGATATACAACGTTCACCCTTATAGG; | SEQ ID NO: 4 |
| VirFLC3F: | ACTGTCctgcagGCTGAAAGAAGAGAATCAGGC; | SEQ ID NO: 5 |
| VirFLC3R | CTATGCatcgatCTCAGCCAAGGGAGTATTGAG; | SEQ ID NO: 6 |
| VirFLC4F: | ACTGTCctgcagCTAGCCAGATGGAGAAGAATAATC; | SEQ ID NO: 7 |
| VirFLC4R: | CTATGCaagcttAAGAGAGTGTGAAGATATACAACG; | SEQ ID NO: 8 |
| VirFLC5F: | ACTGTCctgcagCCGAAGCTGATAATATAGAGATGTC; | SEQ ID NO: 9 |
| VirFLC5R: | CTATGCaagcttGGGTTAAACTGACATAGGTTATTTG. | SEQ ID NO: 10 |

The nucleotides represented in small letter (i.e., not capitalized) correspond to the sequences of the restriction sites used for cloning the PCR amplified fragments.

The cloning strategy was as follows:

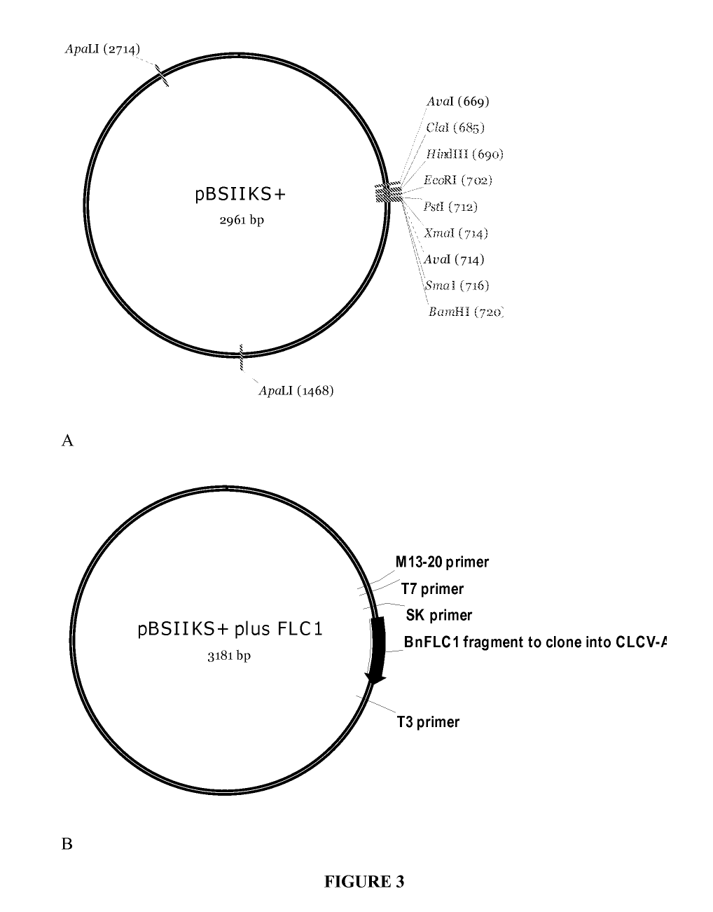

The FLC1 gene fragment of SEQ ID NO: 30 was PCR amplified and cloned into pBSIIKS+ at the ERI/HindIII sites (FIG. 3).

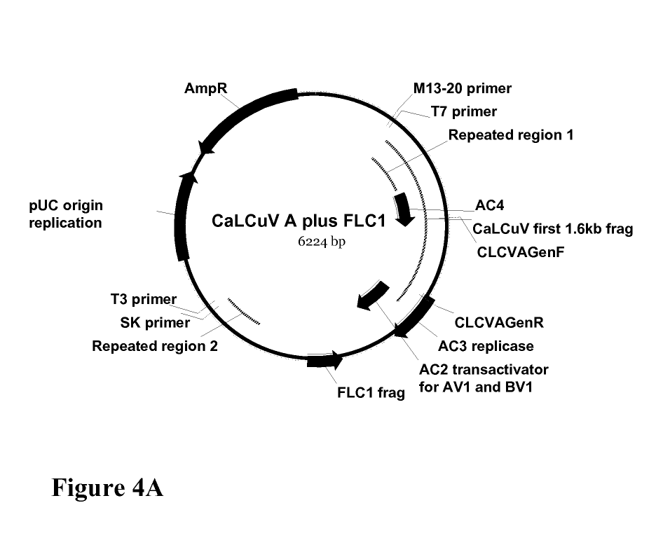

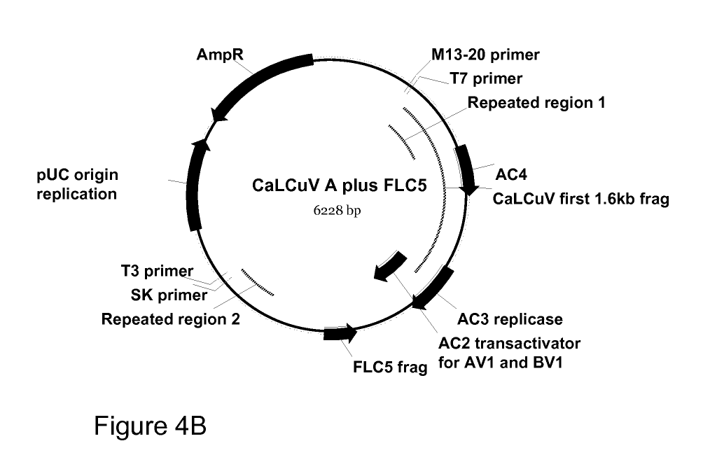

The SmaI/HincII fragment from this modified pBSIIKS+Plus FLC1 vector was removed and cloned into the vector provided by the U. of Florida, CaLCuV-A component vector (partially digested), at HincII/ScaI sites to generate CaLCuV A plus FLC1. This step replaced the coat protein with the FLC1 fragment (FIG. 4A). Removing the coat protein gene reduced the likelihood that the virus could be transmitted by whiteflies (Robertson, (2004) Annu. Rev. Plant Biol. 55:495-519). As is known to those skilled in the art and as was stated above, the entire viral genome is not required for VIGS. In the present case, the coat protein gene of the viral genome was removed. The coat protein is coded by 755 base pairs of the 2583 base pairs of the viral vector CaLCuV A (FIG. 1A) (29% of the A component). The B component of the viral vector is 2513 base pairs (total of 2583+2513=5096). The coat protein corresponds to 14.8% of the total viral genome.

Then, a PstI/HindIII fragment from the CaLCuV A plus FLC1 vector was removed. The remaining PCR-amplified cDNA BnFLC fragments were inserted at the PstI/HindIII sites of the CaLCuV A plus FLC1 vector (except for BnFLC3) in order to generate a total of four different vectors. The plasmid containing FLC5 is shown in vector map of FIG. 4B. For the BnFLC3 gene, the cDNA fragment was amplified with primers containing the PstI/CIaI sites, and cloned into the PstI/CIaI sites of the CaLCuV A plus FLC1 vector.

For the BnFLC-consensus, a consensus sequence with certain complementarity to all five FLC genes is shown in FIG. 2. This region was selected because it has no similarity or domains that are shared with any other known genes and because it was unique to the Brassica FLC gene family. The region chosen to generate the consensus sequence is unique to the FLC genes and, in this way, silencing of all FLC genes at once can be achieved. This region is not found in BnFLC1-5 fragments used in the viral vectors.

The consensus sequence was identified, divided into two long oligonucleotides, PCR amplified with primers containing the PstI/CIaI restriction sites and cloned at the PstI/CIaI sites of the previously modified CaLCuV A plus FLC1 component vector. A modified protocol described in Holowachuck and Ruhoff, 1995 (“Efficient Gene Synthesis by Klenow Assembly/Extension-Pfu Polymerase Amplification (KAPPA) of Overlapping Oligonucleotides” PCR Methods Appl. (1995) 4:299-302) was used. The consensus sequence was synthesized by an initial overlap annealing of single-stranded long oligonucleotides that span the length of the designed sequence. Then, the assembly/extension or ‘fill-in’ of the overlapping oligonucleotides was performed with the Taq DNA Polymerase, as well as the selective amplification of full-sized gene product with the thermostable Taq DNA Polymerase and short terminal oligonucleotide primers. That is why two sets of primers were required: one set of single-stranded long oligonucleotides for the overlap annealing and one set for the selective amplification of full-sized consensus gene product (Table 2).

TABLE 2
Primers used for BnFLC-Consensus:

| | | |
|---|---|---|
| ConsensusFLCLong5′: | GCCCTCTCCGTAACTAGAGCTAGGAAGACAGAACTAATGTTG | SEQ ID NO: 11 |
| | AAGCTTGTGATAGCC | |
| ConsensusFLCLong3′: | TGGTTCTCTTCTTTCAGCAAATTCTCCTTTTCTTTGAGGCTAT | SEQ ID NO: 12 |
| | CACAAGCTTCAACATTAGTTCTG | |
| ConsensusFLCF: | ACTGTCctgcagGCCCTCTCCGTAACTAGAGC; | SEQ ID NO: 13 |
| ConsensusFLCR: | CTATGCatcgatTGGTTCTCTTCTTTCAGCAA | SEQ ID NO: 14 |

The vector with the FLC consensus fragment is shown in FIG. 5.

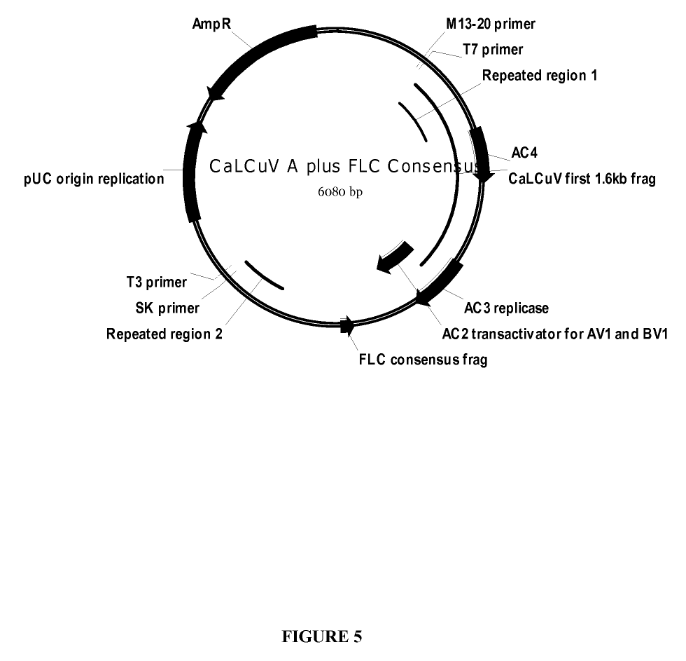

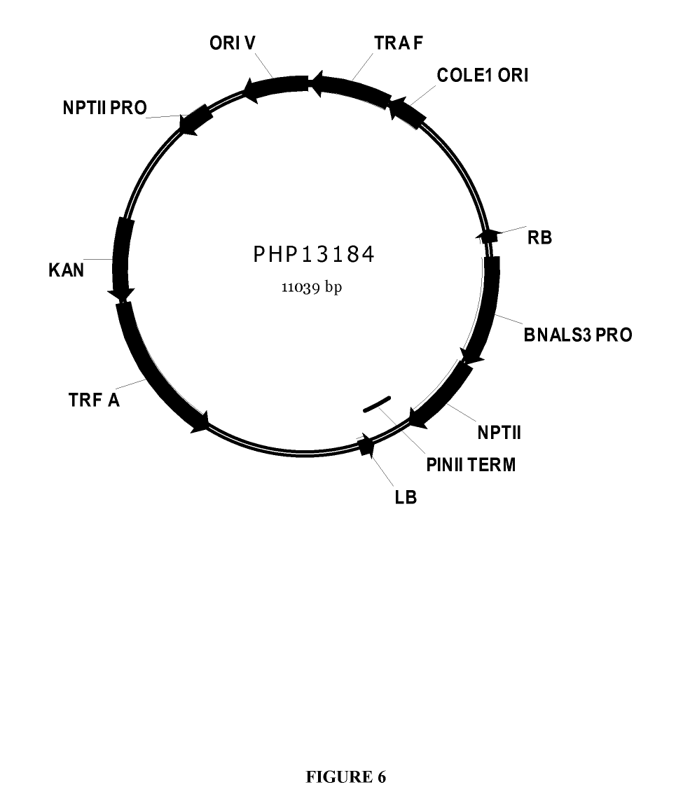

The vector PHP13184 was also used to clone the viral sequences, as shown in FIG. 6.

A fragment from the Left to Right Borders was removed by performing a restriction enzyme digestion with BgIII, and blunt-ended with Klenow. This vector was used as a backbone, and the viral vectors (pUC19 derivative vectors: CaLCuV A, CaLCuV A plus FLC1, CaLCuV plus FLC5, CaLCuV plus FLC-Consensus) containing the FLC sequences were digested with PvuIII, the fragments purified and cloned into the PHP13184 vector. In this way the origins of replication were intact and the T-DNA sequence was removed. The removal of the T-DNA reduced the probability of generating stably transformed plants.

The BnPDS (phytoene desaturase) gene was used as an internal control to test the efficiency of infection and silencing. Silencing of PDS leads to the inhibition of carotenoid synthesis, causing the plants to exhibit a visible photo-bleached phenotype. PCR primers containing the PstI/CIaI restriction sites were used for cloning the PDS fragment into the PstI/CIaI sites of the CaLCuV A plus FLC1 vector. Primers are shown in Table 3.

TABLE 3
Primers for cloning PDS fragment

| | | |
|---|---|---|
| PDSF: | ACTGTCctgcagGATATACCAAGGCCAGAGCTAGA | SEQ ID NO: 45 |
| PDSR: | CTATGCatcgatTCCCAAGTTCTCCAAATAAGTTC | SEQ ID NO: 46 |

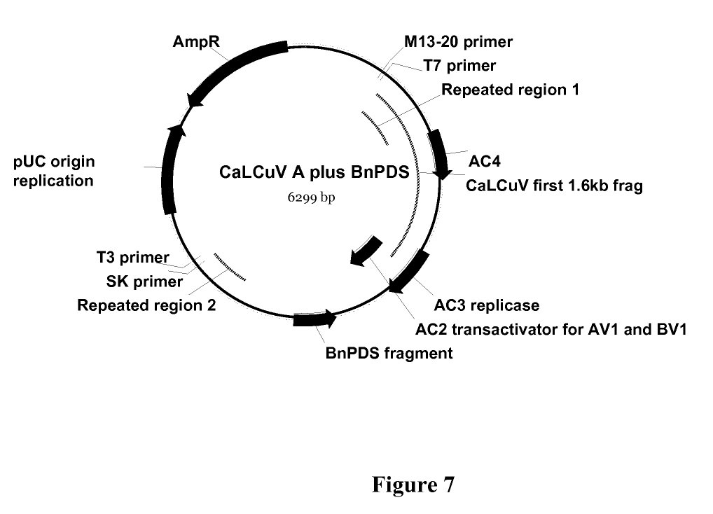

The vector is shown in FIG. 7. Table 8 lists the sequence identification numbers and a brief description of the sequences.

### Example 2

### Plant Transformation

Both the pUC19 derivative and the PHP13184 derivative vectors can be used to transiently transform Brassica plants. Winter Brassica napus was transformed by particle bombardment using the protocols for biolistic transformation of Brassica napus essentially as described in U.S. Pat. No. 6,297,056. However, instead of bombarding microspores, seedlings were bombarded. Seeds from the winter line ‘Express’ were germinated on B5+GA media (see, Table 4). Approximately two weeks after germination, the seedlings were bombarded with the above mentioned vectors. The concentration of DNA used in the bombardment was 3 pg/bp/preparation (each preparation of 50 ul contained 3 pg DNA per basepair). The actual concentration of DNA was dependant on the DNA fragment length. The height of the shelf was 20 cm. The distance between particles and plant tissue was 8 cm. The metal particles were gold (0.6 um in diameter) and the pressure was 900 psi.

After bombardment, the seedlings were kept for approximately one more week on the B5+GA plates, and then transferred to soil and placed in environmentally controlled growth chambers. The growth chamber conditions were 16 hours of light at 22° C. and 8 hours darkness at 18° C. The seedlings were watered daily and fertilized every other day.

TABLE 4
Recipe for B5 + GA + 2% Sucrose

| B5 10x Stock (use 100 ml/L of | | |
|---|---|---|
| B5 10x stock to make 1 L B5 media) | | |
| Stock Ingredients | 4 | L |
| Potassium Nitrate (KNO₃) | 100.0 | g |
| Magnesium Sulfate (MgSO₄—7H₂O) | 10.00 | g |
| Calcium Chloride Dihydrate (CaCl₂—2H₂O) | 30.00 | g |
| Ammonium Sulphate ((NH₄)2SO₄ | 5.36 | g |
| Sodium Phosphate Monobasic (NaH₂PO₄—H₂O) | 6.00 | g |
| Sequestrene, 330 (10% Iron Chelate) | 1.6 | g |
| Bring up the volume to 4 L with filtered water. | | |
| B5 + GA + 2% SUCROSE | | |
| Media ingredients | 2.5 | L |
| B5 10x stock | 250 | ml |
| Sucrose (2%) | 50 | g |
| Gibberellic Acid (0.1 mg/L) | 250 | ul |
| | (stock: 0.02 g/20 ml | |
| | EtOH = 1 mg/ml) | |
| Agar (Sigma # A1296) | 15 | g |
| pH sol'n to 5.8 and bring up the volume to | | |
| 2.5 L with filtered water. | | |
| Autoclave and then pour 35 ml per plate. | | |

Vector combinations used were:
a) CaLCuV A plus FLC1+CaLCuV-B (CaLCuV A plus FLC1 comprises the FLC1 gene fragment)
b) CaLCuV A plus FLC 2 to 5+CaLCuV-B (4 different combinations, one for each of FLC2, FLC3, FLC4, and FLC5)
c) CaLCuV A plus FLC-Consensus+CaLCuV-B
d) CaLCuV-A plus PDS+CaLCuV-B
e) PHP13184::CaLCuV A plus FLC1+PHP13184::CaLCuV-B (CaLCuV-A plus FLC1 comprises the FLC1 gene fragment)
f) PHP13184::CaLCuV A plus FLC 2 to 5+PHP13184::CaLCuV-B (4 different combinations, one for each of FLC2, FLC3, FLC4, and FLC5)
g) PHP13184::CaLCuV-A plus FLC-Consensus+PHP13184::CaLCuV-B
h) PHP13184::CaLCuVA plus PDS+PHP13184::CaLCuV-B

After biolistic transformation, and in order to determine the presence and dispersion of viral DNA A and B components, the following PCR primers were designed to test for viral movement. The CLCV-BGenF and CLCV-BGenR primers target the B component from the BV1 gene to the BC1 gene. That is, they span both genes (see, FIG. 1B). The CLCV-AGenF and CLCV-AGenR primers target the A component from the AC4 gene to the AC3 gene. That is, they span both genes (see, FIG. 1A). The conditions for PCR were as follow: 32 cycles for 30 seconds at 95° C., 30 seconds at 58° C., 40 seconds at 72° C. Both leaves and floral buds were assayed.

TABLE 5
Primers to test for viral movement

| | | |
|---|---|---|
| CLCV-BGenF: | GGATCTACCACGATATCTAATAGGC; | SEQ ID NO: 15 |
| CLCV-BGenR: | ACAGAGTTAGCGACACAAATGTG; | SEQ ID NO: 16 |
| CLCV-AGenF: | AATAAAGACGACGTCTACCACAAC; | SEQ ID NO: 17 |
| CLCV-AGenR: | TCTTGTGCTGTGCTTTGATAGAG.. | SEQ ID NO: 18 |

### Example 3

### Gene Down-Regulation and Flowering Phenotype

Several approaches have been employed to achieve gene silencing in Brassica, but none using VIGS vectors. Here, with the use of biolistic transformation and VIGS technology, a silencing system was developed in Brassica to determine gene function and to induce flowering without vernalization in winter Brassica lines.

In a first biolistic transformation experiment, the VIGS constructs CaLCuV-A plus FLC1, CaLCuV-A plus FLC2, CaLCuV-A plus FLC3, CaLCuV-A plus FLC4, CaLCuV A plus FLC5, CaLCuV-A plus FLC Consensus; CaLCuV-A plus PDS, and CaLCuV-B were used to transform 17-days old seedlings. Initially, 12 seedlings were transformed with each vector following the protocol described above. Following transformation, the plants were maintained for 10 days on Petri dishes and then transferred to soil. After one week in soil, plants transformed with the control Phytoene Desaturase (PDS) containing-vector started to develop a visible phenotype as described in Turnage, et al., (The Plant Journal (2002) 30(1):107-114). 40% of the PDS transformed plants developed yellow and/or chlorotic areas corresponding to PDS down-regulation. However, because downregulation is not uniform, the phenotypes varied. Three plants transformed with the FLC5 gene developed a mild phenotype (that is, small, dispersed yellow and/or chlorotic areas) suggesting damage from bombardment or dispersion of the virus.

In order to confirm the presence and dispersion of the virus, the set of primers described above were designed to determine if the viral DNA (A and B components) was present and spreading systemically. Samples were taken from new leaves (two and a half weeks post bombardment). The results indicated that the viral DNA was present and that it was systemically spreading. The identity of all PCR products was confirmed by sequencing.

The next step was to begin reverse transcription-polymerase chain reaction (RT-PCR) testing (transcription) for PDS and BnFLC down-regulation at different times post-transformation. Table 6 lists the primers used:

TABLE 6
Primers used to assay down-regulation

| | | |
|---|---|---|
| BnFLC1F | GCCAGATGGAGAAGAGTAATCTT | SEQ ID NO: 19 |
| BnFLC1R | GAGCCGGAGAGAGAGTATAGATTAT | SEQ ID NO: 20 |
| BnFLC2F | CTAGCCAGATGGAGAAGAATAATC | SEQ ID NO: 21 |
| BnFLC2R | GATATACAACGTTCACCCTTATAGG | SEQ ID NO: 22 |
| BnFLC3F | GCTGAAAGAAGAGAATCAGGC | SEQ ID NO: 23 |
| BnFLC3R | CTCAGCCAAGGGAGTATTGAG | SEQ ID NO: 24 |
| BnFLC4F | CTAGCCAGATGGAGAAGAATAATC | SEQ ID NO: 25 |
| BnFLC4R | AAGAGAGTGTGAAGATATACAACGC | SEQ ID NO: 26 |
| BnFLC5F | CCGAAGCTGATAATATAGAGATGTC | SEQ ID NO: 27 |
| BnFLC5R | GGGTTAAACTGACATAGGTTATTTG | SEQ ID NO: 28 |
| PDSF | GATATACCAAGGCCAGAGCTAGA | SEQ ID NO: 29 |
| PDSR | TCCCAAGTTCTCCAAATAAGTTC | SEQ ID NO: 39 |

Those plants having shown systemic spreading of the virus also showed gene down-regulation when tested by semi-quantitative RT-PCR. In the case of the PDS transformed plants, the down-regulation was 70% (as determined by densitometry analysis). In the case of the BnFLC5 transformed plants, the down-regulation was from 40 to 75% in 7.5-weeks old plants (5.5 weeks after transformation). Downregulation of BnFLC1 to 4 was not significant in this particular experiment. However, these constructs, or similar constructs, may work in other experiments.

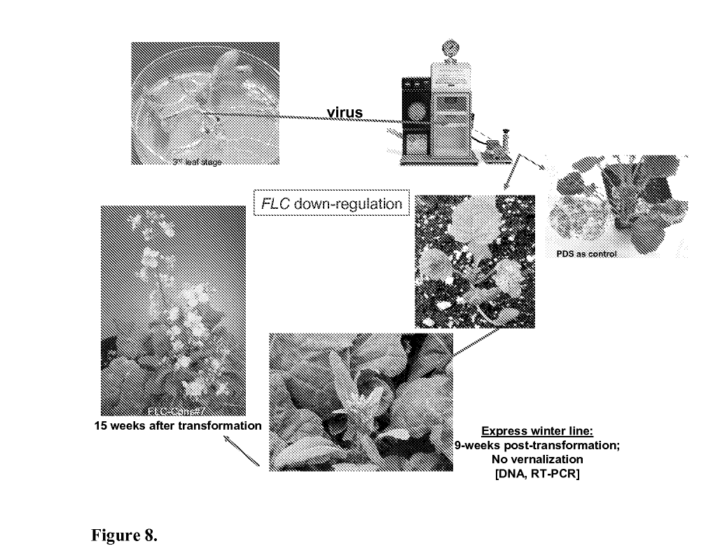

However, after 8 weeks post-transformation (10-weeks old plants), none of the plants had flowered. The plants were left in the growth chambers (under non-vernalization conditions) and the phenotype documented. In the case of the PDS transformed plants, one plant developed a strong phenotype (yellow/chlorotic tissue), and one plant a medium phenotype. From this phenotype, and from PCR reactions performed to detect the viral genome, it was determined that the virus became systemic after 8 weeks post-transformation in canola (FIG. 8). Similar observations were found for the BnFLC5 transformed plants. That is, a strong phenotype related to viral infection was observed after 8 weeks post-transformation (10-weeks old plants) and correlated with the virus systemically spreading. Down-regulation of the FLC5 gene, at this stage, reached 37% (as determined by densitometry analysis). However, none of the BnFLC5 transformed plants flowered after 24 weeks post-transformation.

After 12 weeks post-transformation (14 weeks-old plants), a FLC Consensus transformed plant (plant ConsA#7) began to bolt (FIG. 8). In order to determine if the flowering phenotype was associated with the presence of the viral DNA and its systemic spreading, a series of PCR reactions were performed in different plant tissues to detect the viral A and B components. Floral buds, rosette leaves, and cauline leaves were assayed. The results showed that the viral DNA was more abundant in leaves of the FLC Consensus transformed plant than in floral buds, where it could not be detected. (See, also, Laurent Corbesier, et al., Science (2007) 316:1030-1033). This is expected as CaLCuV has been shown not to infect meristematic tissue (Peele, et al., (2001) The Plant Journal 27(4):357-366.). This result indicated that the virus was present and that it was spreading throughout the FLC-Consensus transformed plant.

The BnFLC1-5 gene expression was determined by semi-quantitative RT-PCR. BnFLC2-5 gene expression was down-regulated in floral buds and rosette leaves of the FLC-Consensus transformed plant, when compared to control plants. BnFLC1-5 gene expression was down-regulated to a greater extent in cauline leaves. Also, the greatest down-regulation was for BnFLC3 in cauline leaves (77.5%), while the least was for BnFLC5 in rosette leaves (9%). These results were corroborated by densitometry analysis, and suggest that in Brassica, flowering can be achieved by partial down-regulation of multiple BnFLC genes.

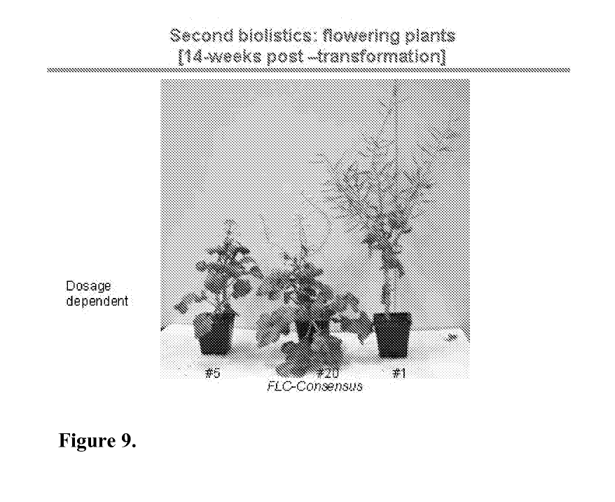

In order to corroborate all previous results, the biolistic transformation experiments were repeated using the PHP13184 set of vectors (i.e., PHP13184::CaLCuVA1, PHP13184::CaLCuVA1+FLC2,PHP13184::CaLCuVA1+FLC3, PHP13184::CaLCuVA1+FLC4, PHP13184::CaLCuVA1+FLC5 and CaLCuVB). Since both sets of vectors are direct DNA vectors, they were expected to work in a similar fashion, which they did. Twenty-eight seedlings were transformed for each construct using the same biolistic method referenced previously. Following transformation, the plants were kept for 10 days in the Petri dishes and then transferred to soil. After one week in soil, the plants developed viral symptoms. The plants were tested for the systemic spreading of the viral DNA in order to correlate flowering with gene down-regulation. The result indicated that the virus was replicating systemically. After 9-weeks post-transformation with FLC-Consensus, 5 plants out of 28, (ConsB#1, ConsB#5, ConsB#6, ConsB#16 and ConsB#20) flowered, indicating that flowering in the Express winter line was achieved by partial down-regulation of multiple BnFLC genes (FIG. 9). The timing for flowering is comparable to that of spring Brassica napus. In summary, we demonstrated that winter lines were induced to flower without vernalization under similar conditions as those used for spring lines.

Table 7 shows the results from the densitometric analysis of FLC1-5 gene expression in BnFLC Consensus transformed plants as determined by semi-quantitative RT-PCR. Leaves and floral buds were assayed. Values are expressed as % of gene expression when compared to untransformed plants (taken as 100% expression). Only some T0 plants that flowered after transformation (without vernalization) are listed.

TABLE 7
Densitometric analysis of the plants that flowered after
transformation with the BnFLC-Consensus vector

| | Gene | | | | |
|---|---|---|---|---|---|
| Plant# | FLC1 | FLC2 | FLC3 | FLC4 | FLC5 |
| ConsB#1 | 72 | 73 | 33 | 57 | 48 |
| ConsB#5 | 100 | 89 | 62 | 67 | 38 |
| ConsB#16 | 82 | 79 | 68 | 67 | 66 |
| ConsB#20 | 67 | 74 | 64 | 57 | 57 |

Percent of gene expression when compared to untransformed plants

### Example 4

### Confirmation of No Viral DNA Integration into the Plant Genome

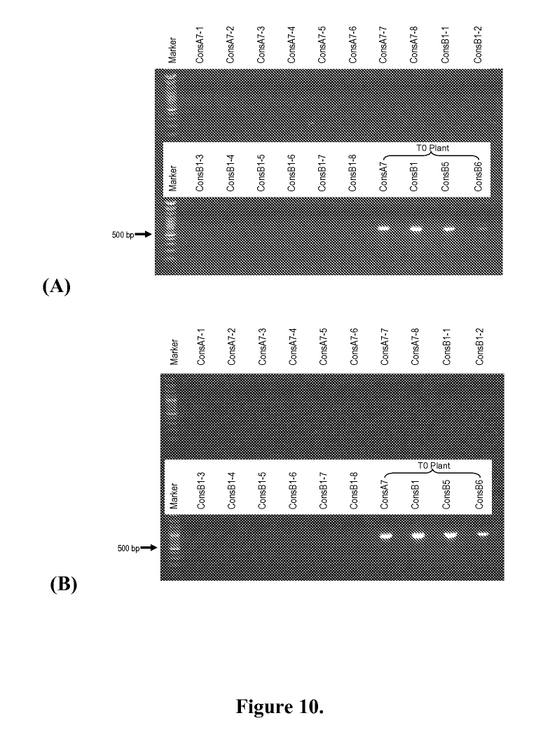

To determine whether viral DNA was integrated into the plant genome during biolistic transformation, sixteen T1 plants from the TO winter Brassica napus FLC-Consensus transformed plants ConsA#7 and ConsB#1 (bombardment experiments A and B, respectively, with FLC-consensus sequence) that flowered without vernalization were investigated, eight T1 plants from each of ConsA#7 and ConsB#1. Plant material from four-week old seedlings was collected and analyzed. After isolating genomic DNA and performing PCR reactions under the conditions described above, none of the T1 plants contained viral DNA (A or B components) as shown in FIG. 10. In addition, none of these T1 plants flowered, when grown under the same conditions as the T0 transformed plants, indicating that no FLC gene down-regulation was occurring in the T1 generation.

These results confirm that the viral DNA was not integrated into the meristematic cells or germ line and that it was not transmitted to the next generation. These results agree with published results indicating that the CaLCuV DNA does not infect meristems, and thus can not be transmitted to the next generation (Peele, et al., (2001) The Plant Journal 27(4):357-366).

### Example 5

### Variations of the Method

The results described above indicate that flowering in winter Brassica napus was achieved without vernalization by simultaneous down-regulation of multiple FLC genes (BnFLC1-5) through VIGS technology. In the experiment described above, downregulation of BnFLC5 alone did not induce flowering without vernalization. It is possible that a different sized fragment (longer or shorter) or different genic regions of the fragment of BnFLC5 can induce flowering without vernalization. Further, use of different methods to transform Brassica with BnFLC5 may also work.

Further, a combination of two, three, or four different FLC fragments may also work. Further still, different sized fragments, either alone or in combination, may also work. The fragments can be longer or shorter than those disclosed here.

Although the examples describe the down-regulation of FLC to reduce or eliminate vernalization, the down regulation of other genes involved in the vernalization process are also included within the scope of the invention. He and Amasino, (2005) Trends in Plant Science 10(1):30-35 describes the vernalization pathway in Arabidopsis. Genes involved in the PAF1 complex (for example ELF7, ELF8 and VIP4) activate FLC expression. Downregulation of a gene or genes in the PAF1 complex may result in reduced vernalization or no vernalization. Further, the FRI and FRL1 genes are believed to up-regulate FLC. Accordingly, downregulation of FRI and FRL1 may reduce or eliminate the vernalization requirement. Finally, VIP3, EFS and PIE1 are also thought to regulate FLC expression. Downregulation of VIP3, EFS and PIE1 may reduce or eliminate the vernalization requirement. Other genes that regulate the vernalization pathway are also included in the scope of this invention. Further the downregulation of a combination of FLC with PAF1, FRI, FRL1, VIP3 or other genes involved in the vernalization process may also work, and are included within the scope of the invention. In wheat, the down regulation of VRN2 is may reduce or eliminate the vernalization requirement.

Based on the fact that all known viroids involve common processes to infect a plant and to propagate, it is to be understood that the aforementioned observations are not specific to cabbage leaf curl virus (CaLCuV) and include other viruses that infect plant hosts. For example, Tomato Golden Mosaic Virus, and among Geminiviruses: Maize streak virus, Beet curly top virus, Bean golden mosaic virus and Tomato pseudo-curly top virus.

Further, based on the fact that RNA-based gene silencing is known to occur across many plant species, it is to be understood that the aforementioned observations are not specific to Brassica, and include other plant species. For example, this technology can be used to reduce or eliminate the vernalization requirement in winter wheat, winter rye, barley, and ryegrass, to name a few.

### Example 6

### Method for Large Scale Commercial Production of Winter Plants that do not Require Vernalization

Methods of introducing vectors to a large population of plants are known to those skilled in the art. For example, Uhde, et al., (2005) Archives of Virology 150(2):327-340 report that viral vectors can be inoculated by gentle abrasion of the surfaces of leaves with carborundum and plasmid DNA. After inoculation, the surface of the leaves are rinsed to remove carborundum and excess DNA. This is a method that can be used to inoculate the CaLCuV modified vectors and to induce flowering without vernalization.

A different method involves “Agrodrench” (Ryu, et al., (2004) The Plant Journal 40(2):322-331). Here, a mixture of Agrobacterium strains containing the viral vectors are suspended in Agrobacterium inoculation buffer and the crown portion of each plant is drenched with the Agrobacterium suspension. The accumulation of the viral DNA induces the silencing of the FLC genes and subsequent induction of flowering without vernalization or with a reduced requirement for vernalization.

### Example 7

### Kits

Kits are provided for (i) silencing vernalization genes, for example the FLC gene, in plants, (ii) amplifying a vernalization gene or a fragment thereof, (iii) assaying for viral movement in a plant and (iv) assaying for down-regulation of a vernalization gene, for example the FLC gene, in a plant. The kits include the primers and sequences disclosed above and instructions as taught in the present invention. The kits may also include buffers and other reagents. Further, the kit may also include a combination of the above kits.

While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

All cited documents are incorporated herein by reference.

TABLE 8
Summary of sequence identification numbers (SEQ ID NO)

| SEQ ID NO: | DESCRIPTION |
|---|---|
| 1, 2, 3, 4, 5, 6, 7, 8, 9, 10 | Primers to amplify BNFLC 1-5 fragments used |
| | in viral vectors (See Table 1) |
| 11, 12, 13, 14 | Primers to amplify BNFLC consensus fragment |
| | used in viral vectors (see Table 2) |
| 15, 16, 17, 18 | Primers used to test viral migration (see Table 5) |
| 19, 20, 21, 22, 23, 24, 25, 26, 27, 28 | Primers to assay for down regulation of |
| | endogenous vernalization genes (see Table 6) |
| 30, 31, 32, 33, 34 | BNFLC1-5 fragments used in viral vectors |
| 35 | BNFLC consensus fragment sequence used in |
| | viral vectors (see FIG. 2) |
| 36, 37, 38 | complete nucleotide sequence of viral vectors: |
| | CaLCuV A Plus FLC1, CaLCuV A Plus FLC5, |
| | CalCuV A Plus FLC Consensus (see FIGS. |
| | 4A, 4B, and 4C) |
| 40, 41, 42, 43, 44 | Fragments of FLC1-5 shown in FIG. 2 used |
| | to identify the consensus sequence (SEQ ID NO: 35) |
| 45, 46 | Primers used for cloning PDS fragment (see Table 3) |
| 29, 39 | Primers used to assay down regulation of PDS (see Table 6) |

What is claimed is:

1. A DNA construct comprising a viral silencing vector, wherein silencing of an endogenous vernalization gene is induced when the DNA construct is introduced into a winter plant that comprises the endogenous vernalization gene.

2. The DNA construct of claim 1 wherein the vernalization gene is selected from the group consisting of flowering locus C (FLC), frigida (FRI), vernalization independence 3 (VIP 3), frigida-like 1 (FRL1), FRI-related activators, photoperiod independent early flowing (PIE1), early flowering in short days (EFS), genes related to the PAF1 complex, early flowering 7 (ELF7), early flowering 8 (ELF8), vernalization independence 4 (VIP4), FLC-related repressors, flowering locus M (FLM); MADS affecting flowering 2 (MAF2), MADS affecting flowering 3 (MAF3), MADS affecting flowering 4 (MAF4), ATX1 (Arabidopsis trithorax 1), wheat vernalization gene 2 (VRN2), structural homologues thereof, functional homologues thereof, and similar sequences.

3. The DNA construct of claim 1 wherein the vernalization gene is an FLC gene.

4. The DNA construct of claim 1 wherein the viral genome is a geminivirus genome.

5. The DNA construct of claim 4 wherein the geminivirus genome is a cabbage leaf curl virus (CaLCuV) genome.

6. The DNA construct of claim 4 wherein the geminivirus genome comprises a nucleotide sequence of GenBank accession number U65529 or U65530, or a portion thereof sufficient to effect VIGS.

7. The DNA construct of claim 3 wherein the FLC gene comprises a fragment of a nucleotide selected from the group consisting of GenBank accession numbers: AY036888 (BnFLC1), AY036889 (BnFLC2), AY036890 (BnFLC3), AY36891 (BnFLC4) and AY036892 (BnFLC5).

8. The DNA construct of claim 1 wherein the second nucleotide comprises a fragment amplified from primer pairs selected from the group consisting of: SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12 and SEQ ID NO: 13 and SEQ ID NO: 14.

9. The DNA construct of claim 1 wherein the second nucleotide comprises a fragment selected from the group consisting of SEQ ID NO: 30, SEQ ID NO: 31, SEQ ID NO: 32, SEQ ID NO: 33, SEQ ID NO: 34 and SEQ ID NO: 35.

10. The DNA construct of claim 1 wherein the DNA construct is that shown in FIG. 4 or FIG. 5, the sequences of which are provided in SEQ ID NO: 36, 37 and 38.

11. The DNA construct of claim 1 wherein the plant is selected from the group consisting of winter Brassica, Arabidopsis, wheat, barley, and ryegrass.

12. The DNA construct of claim 11 wherein the plant is winter Brassica.

13. The DNA construct of claim 1 wherein the plant flowers with a reduced requirement for vernalization compared to a corresponding plant which does not contain the vector, or wherein the plant flowers without the need for vernalization.

14. The DNA construct of claim 1 wherein the plant flowers without vernalization.

15. A method of reducing or eliminating the requirement for vernalization in a winter plant comprising an endogenous vernalization gene, the method comprising the steps:
introducing the DNA construct of claim 1 into the winter plant; and growing the winter plant in plant growth conditions, wherein silencing of the endogenous vernalization gene is induced and wherein the silencing of the endogenous vernalization gene reduces or eliminates the requirement for vernalization in the winter plant compared to a corresponding winter plant without the DNA construct.

16. The method of claim 15 wherein the vernalization gene is selected from the group consisting of flowering locus C (FLC), frigida (FRI), vernalization independence 3 (VIP 3), frigida-like 1 (FRL1), FRI-related activators, photoperiod independent early flowing (PIE1), early flowering in short days (EFS), genes related to the PAF1 complex, early flowering 7 (ELF7), early flowering 8 (ELF8), vernalization independence 4 (VIP4), FLC-related repressors, flowering locus M (FLM); MADS affecting flowering 2 (MAF2), MADS affecting flowering 3 (MAF3), MADS affecting flowering 4 (MAF4), ATX1 (Arabidopsis trithorax 1), wheat vernalization gene 2 (VRN2) structural homologues thereof, functional homologues thereof, and similar sequences.

17. The method of claim 16 wherein the vernalization gene is FLC.

18. The method of claim 15 wherein the step of introducing the DNA construct is by transient transformation.

19. The method of claim 15 wherein the step of introducing the DNA construct is by stable transformation.

20. The method of claim 15 wherein the plant is selected from the group consisting of winter Brassica, Arabidopsis, wheat, barley and ryegrass.

21. The method of claim 20 wherein the plant is winter Brassica.

22. The method of claim 15 wherein the step of introducing the viral silencing vector is selected from the group consisting of particle bombardment, Agrobacterium-mediated transformation, syringe inoculation, Agrodrench, abrasion of plant surfaces and plasmid inoculation.

23. The method of claim 15 wherein the vernalization requirement is eliminated.

24. A method of silencing expression of an endogenous plant gene in a Brassica plant cell, comprising introducing a DNA construct into the plant cell, wherein the DNA construct comprises (i) a first nucleotide comprising at least a fragment of a CaLCuV genome sufficient to effect VIGS and (ii) a second nucleotide comprising a fragment of the endogenous plant gene, or a fragment similar thereto, wherein introduction of the construct in the plant cell results in silencing of the endogenous gene in the plant cell.

25. The method of claim 24 wherein the endogenous gene regulates male fertility.
